(19) United States
(12) Patent Application Publication
(10) Pub. No.: US 2018/0100023 A1
(43) Pub. Date: Apr. 12, 2018

(54) Composition Enriched in Anti-A and /or Anti B Polyclonal Immunoglobulins

(71) Applicant: Laboratoire Francais du Fractionnement et des Biotechnologies, Les Ulis (FR)

(72) Inventors: Philippe PAOLANTONACCI, Gif Sur Yvette (FR); Catherine DE COUPADE, Gif sur Yvette (FR); Stephane BOYER, Pecqueuse (FR)

(73) Assignee: Laboratoire Francais du Fractionnement et des Biotechnologies, Les Ulis (FR)

(21) Appl. No.: 15/568,781

(22) PCT Filed: May 6, 2016
(86) PCT No.: PCT/EP2016/060158

§ 371 Date: Oct. 23, 2017

(30) Foreign Application Priority Data
May 7, 2015 (FR) 1554122

(51) Int. Cl. C07K 16/34; C07K 16/06; G01N 33/80; C07K 1/22

(52) U.S. Cl. CPC C07K 16/34; C07K 16/065; B01D 15/3804; C07K 1/22; C07K 2317/21; G01N 33/80

(57) ABSTRACT

The present invention relates to a composition highly enriched in anti-A and/or anti-B polyclonal immunoglobulins, comprising human polyclonal immunoglobulins, characterized in that at least 80% by weight of the human polyclonal immunoglobulins present in the composition are human anti-A and/or anti-B polyclonal immunoglobulins. The invention also relates to a method for preparing such a composition, and to the use of the composition for preparing a positive control that can be used in a method for assaying anti-A and/or anti-B activity of human normal immunoglobulins, and to use of the composition for determining an individual's blood type.

### FIELD OF THE INVENTION

The present invention is in the field of reference products used to characterize therapeutic polyclonal immunoglobulin concentrates. It relates to a composition that is highly enriched in anti-A and/or anti-B polyclonal immunoglobulins, to a method for preparing such a composition, as well as to the use of the composition for preparing a positive control useful in a method for assaying anti-A and/or anti-B activity of human normal immunoglobulin concentrate, and to use of the composition for determining an individual's blood type.

### PRIOR ART

Human normal immunoglobulins (purified human polyvalent immunoglobulins from plasma pooled from at least 1000 donors) are used to treat a growing number of pathologies. They are used in particular as replacement therapies in primary immunodeficiencies (congenital deficiencies) or secondary immunodeficiencies (chronic lymphocytic leukemia, myeloma, post-bone marrow transplant infections, recurrent bacterial infections in HIV-infected children) characterized by a lack of antibody production. They are also used as an immunomodulatory treatment in various autoimmune pathologies, such as idiopathic thrombocytopenic purpura (ITP), Birdshot's retinochoroiditis, Guillain-Barre syndrome, multifocal motor neuropathy (MMN), chronic inflammatory demyelinating polyneuropathies (CIDP), or Kawasaki's disease.

This growing use requires an increasingly greater supply of human normal immunoglobulins. This leads to the use of increasingly large donor pools. A significant proportion of donors are blood group O, and consequently their plasma contains anti-A and anti-B immunoglobulins directed against antigens of blood groups A and B. Furthermore, blood group A donors generally have anti-B immunoglobulins and blood group B donors have anti-A immunoglobulins. Only blood group AB donors do not have anti-A and anti-B immunoglobulins, but these donors are rare.

However, when the proportions of anti-A and anti-B immunoglobulins in human normal immunoglobulins are too high, they are likely to cause accidental hemolysis, which is potentially severe, in treated patients carrying A and/or B antigens.

Consequently, health authorities require human normal immunoglobulins to be tested with regard to blood groups A and B red blood cell agglutination activity, and set limits for this activity. Thus, for a long time, according to the European Pharmacopoeia, human normal immunoglobulins for intravenous administration (IVIG) must not show A and B red blood cell agglutination at a 1:64 dilution of a solution whose initial concentration is 30 g/l (3%), in an indirect antiglobulin test (IAT, also called indirect Coombs test).

However, it was then shown that the IAT test is highly variable and, moreover, is likely to underestimate the anti-A and anti-B activity of human normal immunoglobulins (Thorpe et al. Biologicals. 2005 June; 33(2):111-6). Indeed, the IAT test is based on the use of antiglobulin, i.e., immunoglobulins of animal origin that specifically recognize human immunoglobulins. However, the ability of antiglobulin to agglutinate group A or B red blood cells coated with anti-A or anti-B immunoglobulins is likely to be saturated by the concomitant presence of a high concentration of human immunoglobulins directed against antigens other than those of blood groups A and B. In order to limit this variability, Thorpe and colleagues developed a direct agglutination method, not using antiglobulin, wherein A or B red blood cells treated with papain are brought into contact with two-fold serial dilutions of 5% (50 g/l) human normal immunoglobulin solution, in a microplate with V-shaped wells. After centrifugation, the plate is tilted at an angle of about 70° for about 4-5 minutes. If agglutination occurs, the cell pellet remains stuck to the bottom of the V-shaped well, forming a well-rounded point. Otherwise, the non-agglutinated cell pellet slides along the wall of the V-shaped well, thus generating a droplet-type shape (Thorpe et al. Biologicals. 2005 June; 33(2):111-6).

Although leading to less variable results than the IAT test, this method still lacks positive and negative controls. To make up for this lack, Thorpe and colleagues then developed three human immunoglobulin reference products for calibrating the previously-developed direct agglutination method (Thorpe et al. Vox Sang. 2009 August; 97(2):160-8; Thorpe et al. Pharmeur Bio Sci Notes. 2010 April; 2010(1):39-50; document WHO/BS/08.2091, which can be downloaded from the address http://apps.who.int/iris/handle/10665/69970?locale=fr):
A negative control (fraction 07/308), consisting of 5% (50 g/l) human normal immunoglobulins purified from plasma from only blood group AB donors, whose plasma does not contain anti-A and anti-B immunoglobulins.
A positive control (fraction 07/306), consisting of 5% (50 g/l) human normal immunoglobulins known to have anti-A and anti-B activity superior to the mean of human normal immunoglobulins, but not producing A or B red blood cell agglutination at a 1:64 dilution in the previously-developed direct agglutination method.
A limit positive control (fraction 07/310), consisting of fraction 07/306, to which amounts of murine anti-A monoclonal antibody (BRIC 145) and murine anti-B monoclonal antibody (BGRL 1) were added, so that this control produces A or B red blood cell agglutination at a dilution of at most 1:64 in the previously-developed direct agglutination method.

The tests carried out with these controls showed that said controls can make it possible to standardize anti-A and anti-B immunoglobulin agglutination tests, to verify that the tests used are sufficiently specific and sensitive, and to facilitate the identification of batches of human normal immunoglobulins whose anti-A and anti-B activity exceeds that allowed by health authorities.

Following this work, the three controls mentioned above were adopted as reference reagents by the European Pharmacopoeia, the World Health Organization (WHO) and the Food and Drug Administration (FDA), and the limit of anti-A and anti-B activity of IVIGs in the European Pharmacopoeia was revised: IVIGs now must not show A and B red blood cell agglutination at a 1:64 dilution of a solution whose initial concentration is 50 g/l (5%), in a specific direct agglutination test corresponding to that developed by Thorpe and colleagues (European Pharmacopoeia, chapter 2.6.20 as revised by supplement 7.2 of January 2011).

However, the positive controls mentioned above have their disadvantages, and in particular the limit positive control, which supposedly has anti-A and anti-B activity corresponding to the limit accepted by health authorities. Indeed, as indicated by Thorpe and colleagues, one of the principal flaws of this limit positive control is its limited availability. In particular, due to its insufficient availability, use of this product is to date recommended only for verifying the anti-A and anti-B activity of human normal immunoglobulin concentrates having anti-A anti-B activity superior to that of fraction 07/306 (positive control, 5% IVIG with anti-A and anti-B activity superior to the mean but inferior to the limits set by health authorities) (Thorpe et al. Vox Sang. 2009 August; 97(2):160-8; Thorpe et al. Pharmeur Bio Sci Notes. 2010 April; 2010(1):39-50). Thus, the limited availability of the sole limit positive control existing to date does not allow for its systematic use in anti-A and anti-B immunoglobulin agglutination tests of human normal immunoglobulin concentrates.

Moreover, the anti-A and anti-B immunoglobulins present in this limit positive control are of murine and not human origin, and are in addition monoclonal and not polyclonal, unlike the anti-A and anti-B immunoglobulins present in human normal immunoglobulin concentrates. These two differences may lead to underestimation or overestimation of the anti-A and anti-B activity of human normal immunoglobulins, which is undesirable in both cases.

Therefore, there exists a need for novel positive controls that make it possible to calibrate anti-A and anti-B immunoglobulin agglutination tests and to reliably detect batches of human normal immunoglobulins having anti-A and anti-B activity superior to the limits accepted by health authorities.

With the aim of avoiding accidental hemolysis without reducing the donor pools able to be used, plasma fractionators have developed methods for eliminating anti-A and anti-B immunoglobulins from their human normal immunoglobulin concentrates.

For example, WO01/27623 and WO2007/077365 describe the use of various affinity chromatography supports that specifically bind to anti-A and anti-B immunoglobulins in order to eliminate anti-A and anti-B immunoglobulins from biological compositions, in particular from human normal immunoglobulin concentrates. The unadsorbed fraction not comprising anti-A and anti-B immunoglobulins is collected by percolation, for subsequent treatment and packaging. The anti-A anti-B affinity chromatography column is then regenerated by washes that are then discarded. The anti-A and anti-B immunoglobulin fraction adsorbed during the affinity chromatography step thus today corresponds to a lost fraction.

### SUMMARY OF THE INVENTION

However, in the context of the present invention, the Inventors have shown that the anti-A and anti-B polyclonal immunoglobulin fraction adsorbed on the affinity chromatography column aimed at eliminating anti-A and anti-B immunoglobulins and obtained by combining two column wash fractions may be used to prepare a novel positive control useful for calibrating anti-A and anti-B immunoglobulin agglutination tests and for reliably detecting batches of human normal immunoglobulins having anti-A and anti-B activity superior to the limits accepted by health authorities. This novel positive control has the advantage of being composed of the same human anti-A and anti-B polyclonal immunoglobulins as those likely to be present in varying amounts in human normal immunoglobulin concentrates. Moreover, in view of the increasing amount of human normal immunoglobulins produced each year, this novel positive control could be produced in sufficient amounts to allow its systematic use as limit positive control in all anti-A and anti-B immunoglobulin agglutination tests of human normal immunoglobulin concentrates.

In a first aspect, the present invention thus relates to a composition comprising human polyclonal immunoglobulins, characterized in that at least 80% by weight of human polyclonal immunoglobulins present in the composition are human anti-A and/or anti-B polyclonal immunoglobulins. Advantageously, human polyclonal immunoglobulins represent at least 85% by weight of the total proteins of the composition.

In an advantageous embodiment, at least 80% by weight of human polyclonal immunoglobulins present in the composition are human anti-A polyclonal immunoglobulins. In another advantageous embodiment, at least 80% by weight of human polyclonal immunoglobulins present in the composition are human anti-B polyclonal immunoglobulins. In yet another advantageous embodiment, the composition comprises both human anti-A polyclonal immunoglobulins and human anti-B polyclonal immunoglobulins, and the weight ratio of human anti-A polyclonal immunoglobulins to human anti-B polyclonal immunoglobulins (anti-A:anti-B) is between 1:10 and 10:1.

In an embodiment, the composition comprising both human anti-A polyclonal immunoglobulins and human anti-B polyclonal immunoglobulins is enriched in human anti-A polyclonal immunoglobulins, and thus has a weight ratio of human anti-A polyclonal immunoglobulins to human anti-B polyclonal immunoglobulins (anti-A:anti-B) between 2:1 and 10:1.

In another embodiment, the composition comprising both human anti-A polyclonal immunoglobulins and human anti-B polyclonal immunoglobulins is enriched in human anti-B polyclonal immunoglobulins, and thus has a weight ratio of human anti-A polyclonal immunoglobulins to human anti-B polyclonal immunoglobulins (anti-A:anti-B) between 1:10 and 1:2.

In another embodiment, the composition comprising both human anti-A polyclonal immunoglobulins and human anti-B polyclonal immunoglobulins is balanced in terms of the two types of immunoglobulins, and thus has a weight ratio of human anti-A polyclonal immunoglobulins to human anti-B polyclonal immunoglobulins (anti-A:anti-B) between 3:10 and 7:10, advantageously between 4:10 and 6:10.

The invention also relates to a method for preparing a composition according to the invention, comprising the following steps:
a) adsorbing a batch of human plasma or a human plasma fraction enriched in human polyclonal immunoglobulins on a support grafted with a specific ligand of human anti-A polyclonal immunoglobulins and/or with a specific ligand of human anti-B polyclonal immunoglobulins,
b) storing the unadsorbed fraction for possible later use, and
c) dissociating and collecting the adsorbed fraction.

When at least 80% by weight of human polyclonal immunoglobulins present in the composition according to the invention are human anti-A polyclonal immunoglobulins, the method advantageously comprises the following steps:
a) adsorbing a batch of human plasma or a human plasma fraction enriched in human polyclonal immunoglobulins on a support grafted with a specific ligand of human anti-A polyclonal immunoglobulins, and with a specific ligand of human anti-B polyclonal immunoglobulins,
b) storing the unadsorbed fraction for possible later use,
c) dissociating and collecting the adsorbed fraction,
d) adsorbing the composition obtained in step c) on a support grafted with a specific ligand of human anti-B polyclonal immunoglobulins, and
e) collecting the unadsorbed fraction.

When at least 80% by weight of human polyclonal immunoglobulins present in the composition according to the invention are human anti-B polyclonal immunoglobulins, the method advantageously comprises the following steps:
a) adsorbing a batch of human plasma or a human plasma fraction enriched in human polyclonal immunoglobulins on a support grafted with a specific ligand of human anti-B polyclonal immunoglobulins, and with a specific ligand of human anti-A polyclonal immunoglobulins,
b) storing the unadsorbed fraction for possible later use,
c) dissociating and collecting the adsorbed fraction,
d) adsorbing the composition obtained in step c) on a support grafted with a specific ligand of human anti-A polyclonal immunoglobulins, and
e) collecting the unadsorbed fraction.

In a preparation method according to the invention, the specific ligand of human anti-A polyclonal immunoglobulins is advantageously selected from oligosaccharides representative of group A antigens of type 1, 2, 3 and 4.

In a preparation method according to the invention, the specific ligand of human anti-B polyclonal immunoglobulins is advantageously selected from oligosaccharides representative of group B antigens of type 1, 2, 3 and 4.

In a preparation method according to the invention, the support used in step a) and/or in step d) is advantageously in the form of:
a) particles (in particular polymer particles) grafted with the ligand(s) of interest, or
b) a polymer membrane, the membrane being grafted with the ligand(s) of interest.

The particles are advantageously incorporated into a gel or a resin, which is used as the matrix in an affinity chromatography column. Likewise, the polymer membrane may be included in an affinity chromatography column. The batch of human plasma or the human plasma fraction enriched in human polyclonal immunoglobulins is then adsorbed on the affinity chromatography column, and the adsorbed fraction is eluted and collected.

The particle polymer or membrane polymer is advantageously selected from cellulose and derivatives thereof, agarose, dextran, polyacrylates, polystyrene, polyacrylamide, polymethacrylamide, styrene and divinylbenzene copolymers, or mixtures of these polymers.

In a preparation method according to the invention, the ligand of interest is advantageously grafted on the polymer particles or on the polymer membrane via a spacer.

The invention further relates to a composition able to be obtained by a preparation method according to the invention.

The invention also relates to the use of a composition according to the invention as positive control in a method for assaying the anti-A or anti-B activity of a composition comprising human polyclonal immunoglobulins.

The invention also relates to the use of a composition according to the invention for preparing a positive control and/or a limit positive control intended to be used in a method for assaying the anti-A or anti-B activity of a composition comprising human polyclonal immunoglobulins.

The invention also relates to a method for preparing a positive control intended to be used in a method for assaying anti-A and/or anti-B activity of a composition comprising human polyclonal immunoglobulins, comprising the following steps:
a) providing a composition comprising human polyclonal immunoglobulins having anti-A activity and/or anti-B activity inferior to a given limit value in said assay method, and
b) adding a composition according to the invention.

The invention also relates to a method for preparing a below-limit positive control, a limit positive control or an above-limit positive control, intended to be used in a method for assaying anti-A and/or anti-B activity of a composition comprising human polyclonal immunoglobulins, comprising the following steps:
a) providing a composition comprising human polyclonal immunoglobulins having anti-A activity and anti-B activity inferior to a given limit value in said assay method,
b) adding increasing amounts of a composition according to the invention in which at least 80% by weight of human polyclonal immunoglobulins present in the composition according to the invention are human anti-A polyclonal immunoglobulins and/or of a composition according to the invention in which at least 80% by weight of human polyclonal immunoglobulins present in the composition according to the invention are human anti-B polyclonal immunoglobulins, in order to obtain various compositions enriched in human anti-A and/or anti-B polyclonal immunoglobulins,
c) assaying by said assay method the anti-A and/or anti-B activity of compositions enriched in human anti-A and/or anti-B polyclonal immunoglobulins obtained in step b), and
d) selecting the composition enriched in human anti-A and/or anti-B polyclonal immunoglobulins having anti-A and/or anti-B activity in said assay method inferior to (in order to obtain a below-limit positive control), equal to (in order to obtain a limit positive control), or superior to (in order to obtain an above-limit positive control) said limit value.

The invention further relates to a positive control (below-limit positive control, limit positive control, or above-limit positive control) intended to be used in a method for assaying anti-A and/or anti-B activity of a composition comprising human polyclonal immunoglobulins, able to be obtained by one of the methods according to the invention for preparing a positive control described above.

Finally, the invention relates to the use of a composition characterized in that at least 80% by weight of human polyclonal immunoglobulins present in the composition are human anti-A and/or anti-B polyclonal immunoglobulins or a composition able to be obtained by the method according to the invention in a test for determining an individual's blood type.

### DETAILED DESCRIPTION OF THE INVENTION

In the context of the present invention, the Inventors have shown that the anti-A and/or anti-B polyclonal immunoglobulin fraction adsorbed on the affinity chromatography column aimed at eliminating anti-A and/or anti-B immunoglobulins may be used to prepare a novel positive control useful for calibrating anti-A and/or anti-B immunoglobulin agglutination tests and for reliably detecting batches of human normal immunoglobulins having anti-A and/or anti-B activity superior to the limits accepted by health authorities. This novel positive control has the advantage of being composed of the same human anti-A and/or anti-B polyclonal immunoglobulins as those likely to be present in varying amounts in human normal immunoglobulin concentrates. Moreover, in view of the increasing amount of human normal immunoglobulins produced each year, this novel positive control could be produced in sufficient amounts to allow its systematic use as limit positive control in all anti-A and/or anti-B immunoglobulin agglutination tests of human normal immunoglobulin concentrates.

Definitions

By “antibody” or “immunoglobulin” is meant a molecule comprising at least one binding domain for a given antigen and a constant domain comprising an Fc fragment capable of binding to Fc receptors (FcR).

By “human polyclonal immunoglobulins” is meant a composition of human immunoglobulins directed against numerous distinct antigens, and comprising, for each recognized antigen, multiple distinct immunoglobulins capable of recognizing said antigen, generally at several distinct epitopes. Such human polyclonal immunoglobulins are generally purified from plasma from one donor or, preferably, from several donors (called a donor pool). Therapeutic human normal immunoglobulins are thus purified from plasma generally pooled from at least 1000 donors.

By “human anti-A polyclonal immunoglobulins” or “anti-A immunoglobulins” is meant human polyclonal immunoglobulins that recognize blood group A antigens. “Blood group A antigens” or “A antigens” are characterized by the presence of a trisaccharide comprising N-acetylgalactosamine (abbreviated hereinafter as “GalNAc”) linked to a galactose (abbreviated hereinafter as “Gal”), which is itself linked to a fucose (abbreviated hereinafter as “Fuc”), according to the following sequence: GalNAcα1-3(Fucα1-2)Gal.

This trisaccharide itself may be attached by its central galactose to other sugars, whose number and assembly vary according to the type of A antigen, as indicated in Table 1 below for A antigen types 1 to 4, and to the presence or absence and the nature of a Lewis antigen.

TABLE 1
Structure of various blood group A antigen types.

| | |
|---|---|
| Type 1 | GalNAcα1-3(Fucα1-2)Galβ1-3GlcNAcβ1-3Galβ1-4Glc-R |
| Type 2 | GalNAcα1-3(Fucα1-2)Galβ1-4GlcNAcβ1-3Galβ1-4Glc-R |
| Type 3 | GalNAcα1-3(Fucα1-2)Galβ1-3GalNAcα1-3Galβ1-4GlcNAc-R |
| Type 4 | GalNAcα1-3(Fucα1-2)Galβ1-3GalNAcβ1-3Galα1-4Gal-R |

GalNAc: N-acetylgalactosamine, Fuc: fucose, Gal: galactose, GlcNAc: N-acetylglucosamine, Glc: glucose, R: support (oligosaccharide, glycoprotein, glycolipid).
The trisaccharide that determines group A is indicated in bold.

By “human anti-B polyclonal immunoglobulins” or “anti-B immunoglobulins” is meant human polyclonal immunoglobulins that recognize blood group B antigens. “Blood group B antigens” or “B antigens” are characterized by the presence of a trisaccharide comprising a first galactose linked to a second galactose, which is itself linked to a fucose, according to the following sequence: Galα1-3(Fucα1-2)Gal.

This trisaccharide itself may be attached by its central galactose to other sugars, whose number and assembly vary according to the type of B antigen, as indicated in Table 2 below for B antigen types 1 to 4, and to the presence or absence and the nature of a Lewis antigen.

TABLE 2
Structure of various blood group B antigen types.

| | |
|---|---|
| Type 1 | GalNAcα1-3(Fucα1-2)Galβ1-3GlcNAcβ1-3Galβ1-4Glc-R |
| Type 2 | GalNAcα1-3(Fucα1-2)Galβ1-4GlcNAcβ1-3Galβ1-4Glc-R |
| Type 3 | GalNAcα1-3(Fucα1-2)Galβ1-3GalNAcα1-3Galβ1-4GlcNAc-R |
| Type 4 | GalNAcα1-3(Fucα1-2)Galβ1-3GalNAcβ1-3Galα1-4Gal-R |

GalNAc: N-acetylgalactosamine, Fuc: fucose, Gal: galactose, GlcNAc: N-acetylglucosamine, Glc: glucose, R: support (oligosaccharide, glycoprotein, glycolipid).
The trisaccharide that determines group B is indicated in bold.

By “human plasma fraction enriched in human polyclonal immunoglobulins” is meant any human plasma fraction able to be obtained by fractionation of human plasma and whose percentage by weight of polyclonal immunoglobulins in relation to the total proteins of the fraction is superior to that of human plasma. Such fractions are advantageously obtained by fractionation of plasma pooled from at least 1000 donors. In particular, they may include any part or subpart of plasma having undergone one or more purification steps, in particular supernatant of cryoprecipitated plasma, plasma cryoprecipitate (resuspended or not), fractions I to V obtained by ethanol fractionation (according to the Cohn method or the Kistler and Nitschmann method), supernatant and precipitate obtained after precipitation with caprylic acid and/or caprylate, filtrates, or any fraction enriched in immunoglobulins (chromatography eluates and/or unadsorbed fractions) by chromatographic separation, as described in particular in WO99/64462 and WO02/092632, and more particularly in WO02/092632.

By “specific ligand of human anti-A polyclonal immunoglobulins” is meant a molecule that binds to human anti-A polyclonal immunoglobulins as defined above and that does not bind to other immunoglobulins. In particular, such ligands may be selected from oligosaccharides representing blood group A antigens. By “oligosaccharide representing blood group A antigens” is meant any oligosaccharide comprising the trisaccharide characteristic of blood group A antigens as defined above: GalNAcα1-3(Fucα1-2)Gal. Such an oligosaccharide may further comprise other sugars present in the blood group A antigens defined above. In particular, in addition to the trisaccharide GalNAcα1-3(Fucα1-2) Gal, such an oligosaccharide may also be selected from tetrasaccharides, pentasaccharides and hexasaccharides derived from the type 1, 2, 3 or 4 group A antigens described above and comprising the characteristic trisaccharide GalNAcα1-3(Fucα1-2)Gal:
Tetrasaccharides:
   Type 1: GalNAcα1-3(Fucα1-2)Galβ1-3GlcNAc,
   Type 2: GalNAcα1-3(Fucα1-2)Galβ1-4GlcNAc,
   Type 3 or 4: GalNAcα1-3(Fucα1-2)Galβ1-3GalNAc,
Pentasaccharides:
   Type 1: GalNAcα1-3(Fucα1-2)Galβ1-3GlcNAc β1-3Gal,
   Type 2: GalNAcα1-3(Fucα1-2)Galβ1-4GlcNAc β1-3Gal,
   Type 3: GalNAcα1-3(Fucα1-2)Galβ1-3GalNAc α1-3Gal,
   Type 4: GalNAcα1-3(Fucα1-2)Galβ1-3GalNAc β1-3Gal,
Hexasaccharides:
   Type 1: GalNAcα1-3(Fucα1-2)Galβ1-3GlcNAc β1-3Galβ1-4Glc,
   Type 2: GalNAcα1-3(Fucα1-2)Galβ1-4GlcNAc β1-3Galβ1-4Glc,
   Type 3: GalNAcα1-3(Fucα1-2)Galβ1-3GalNAc α1-3Galβ1-4GlcNAc,
   Type 4: GalNAcα1-3(Fucα1-2)Galβ1-3GalNAc β1-3Galα1-4Gal.

The oligosaccharide may also comprise repeated units of the characteristic trisaccharide GalNAcα1-3(Fucα1-2)Gal, or tetrasaccharides, pentasaccharides and hexasaccharides derived from the type 1, 2, 3 or 4 group A antigens described above.

Advantageously, the specific ligand of the immunoglobulins that recognize blood group A antigens is the characteristic trisaccharide GalNAcα1-3(Fucα1-2)Gal.

By “specific ligand of human anti-B polyclonal immunoglobulins” is meant a molecule that binds to human anti-B polyclonal immunoglobulins as defined above and that does not bind to other immunoglobulins. In particular, such ligands may be selected from oligosaccharides representing blood group B antigens. By “oligosaccharide representing blood group B antigens” is meant any oligosaccharide comprising the characteristic trisaccharide of blood group B antigens such as defined above: Galα1-3(Fucα1-2)Gal. Such an oligosaccharide may further comprise other sugars present in the blood group B antigens defined above. In particular, in addition to the trisaccharide Galα1-3(Fucα1-2)Gal, such an oligosaccharide may also be selected from tetrasaccharides, pentasaccharides and hexasaccharides derived from the group B antigens type 1, 2, 3 or 4 described above and comprising the characteristic trisaccharide Galα1-3(Fucα1-2)Gal:
Tetrasaccharides:
   Type 1: Galα1-3(Fucα1-2)Galβ1-3GlcNAc,
   Type 2: Galα1-3(Fucα1-2)Galβ1-4GlcNAc,
   Type 3 or 4: Galα1-3(Fucα1-2)Galβ1-3GalNAc,
Pentasaccharides:
   Type 1: Galα1-3(Fucα1-2)Galβ1-3GlcNAc β1-3Gal,
   Type 2: Galα1-3(Fucα1-2)Galβ1-4GlcNAc β1-3Gal,
   Type 3: Galα1-3(Fucα1-2)Galβ1-3GalNAc α1-3Gal,
   Type 4: Galα1-3(Fucα1-2)Galβ1-3GalNAc β1-3Gal,
Hexasaccharides:
   Type 1: Galα1-3(Fucα1-2)Galβ1-3GlcNAc β1-3Galβ1-4Glc,
   Type 2: Galα1-3(Fucα1-2)Galβ1-4GlcNAc β1-3Galβ1-4Glc,
   Type 3: Galα1-3(Fucα1-2)Galβ1-3GalNAc α1-3Galβ1-4GlcNAc,
   Type 4: Galα1-3(Fucα1-2)Galβ1-3GalNAc β1-3Galα1-4Gal.

The oligosaccharide may also comprise repeated units of the characteristic trisaccharide Galα1-3(Fucα1-2)Gal, or tetrasaccharides, pentasaccharides and hexasaccharides derived from the type 1, 2, 3 or 4 group B antigens described above.

Advantageously, the specific ligand of the immunoglobulins that recognize blood group B antigens is the characteristic trisaccharide Galα1-3(Fucα1-2)Gal.

Throughout the present description, reference to “a” specific ligand of human anti-A or anti-B polyclonal immunoglobulins includes the possibility of using either a single type of specific ligand of human anti-A or anti-B polyclonal immunoglobulins (i.e., all the ligands grafted onto the support have the same chemical structure), or several distinct types (i.e., different chemical structures) of specific ligands of human anti-A or anti-B polyclonal immunoglobulins. However, it should be understood that each ligand of a given chemical structure that is able to be used is necessarily grafted several times onto the support, in such a way that the human anti-A or anti-B polyclonal immunoglobulins may be retained by the support. The skilled person knows which ligand density must be used in order to allow optimum adsorption of human anti-A or anti-B polyclonal immunoglobulins on the support.

By “positive control” of a method for assaying anti-A and/or anti-B activity of a composition comprising human polyclonal immunoglobulins is meant a human polyclonal immunoglobulin composition comprising human anti-A and/or anti-B polyclonal immunoglobulins.

Regulatory authorities require that human normal immunoglobulin compositions have anti-A activity and anti-B activity inferior to a limit value, set at a given time, but likely to vary over time and according to changes to the requirements set by regulatory authorities.

For a given limit value of anti-A and/or anti-B activity, the expression “positive control” covers three types of positive controls, respectively referred to in the present description as “below-limit positive control,” “limit positive control,” and “above-limit positive control,” each of these positive controls being of interest in a method for assaying anti-A and/or anti-B activity.

By “below-limit positive control” of a method for assaying anti-A and/or anti-B activity of a composition comprising human polyclonal immunoglobulins is meant a positive control comprising an amount of human anti-A and/or anti-B polyclonal immunoglobulins generating in the assay method an activity value inferior to a predefined limit value. Such a limit value may in particular correspond to the maximum anti-A and/or anti-B activity value accepted by health authorities of a country for human normal immunoglobulins intended for administration in humans by any route of administration, in particular by the intravenous or intramuscular or subcutaneous route. Such below-limit positive controls are useful in particular for verifying that the performed assay proceeded satisfactorily, such a control systematically having to have anti-A and/or anti-B activity inferior to the selected limit value. It can also make it possible to determine whether a therapeutic concentrate is close to the maximum limit value accepted by health authorities or is significantly below this limit.

By “limit positive control” of a method for assaying anti-A and/or anti-B activity of a composition comprising human polyclonal immunoglobulins is meant a positive control comprising an amount of human anti-A and/or anti-B polyclonal immunoglobulins generating in the assay method an activity value equal to a predefined limit value. Such a limit value may in particular correspond to the maximum anti-A and/or anti-B activity value accepted by health authorities of a country for human normal immunoglobulins intended for administration in humans by any route of administration, in particular by the intravenous or intramuscular or subcutaneous route. Such a limit positive control is useful in particular for determining whether a therapeutic concentrate has or does not have anti-A and/or anti-B activity inferior to the maximum limit value accepted by health authorities.

By “above-limit positive control” of a method for assaying anti-A and/or anti-B activity of a composition comprising human polyclonal immunoglobulins is meant a positive control comprising an amount of human anti-A and/or anti-B polyclonal immunoglobulins generating in the assay method an activity value superior to a predefined limit value. Such a limit value may in particular correspond to the maximum anti-A and/or anti-B activity value accepted by health authorities of a country for human normal immunoglobulins intended for administration in humans by any route of administration, in particular by the intravenous or intramuscular or subcutaneous route. Such an above-limit positive control is useful in particular for verifying that the performed assay proceeded satisfactorily, such a control systematically having to have anti-A and/or anti-B activity superior to the selected limit value. It can also make it possible to determine whether a therapeutic concentrate is close to the maximum limit value accepted by health authorities or is significantly above this limit.

Human Anti-A or Anti-B Polyclonal Immunoglobulin Compositions

The present invention relates to a composition comprising human polyclonal immunoglobulins, characterized in that at least 80%, at least 81%, at least 82%, at least 83%, at least 84%, advantageously at least 85%, at least 86%, at least 87%, more advantageously at least 88%, at least 89%, even more advantageously at least 90%, at least 91%, at least 92%, at least 93%, at least 94%, even at least 95%, at least 96%, at least 97%, at least 98%, or at least 99% by weight of human polyclonal immunoglobulins present in the composition are human anti-A and/or anti-B polyclonal immunoglobulins.

The percentage by weight of human anti-A and/or anti-B polyclonal immunoglobulins among the total human polyclonal immunoglobulins of a composition comprising purified human polyclonal immunoglobulins may be measured by purifying the composition using affinity chromatography on a column grafted with specific ligands of human anti-A and/or anti-B polyclonal immunoglobulins, and by calculating the ratio between the weight of the immunoglobulins adsorbed on the column and the weight of the total immunoglobulins. If the composition is not purified for immunoglobulins, a preliminary step of purifying the total immunoglobulins then makes it possible to measure the percentage by weight of human anti-A and/or anti-B polyclonal immunoglobulins among the total human polyclonal immunoglobulins.

The compositions according to the invention are preferably purified, and human polyclonal immunoglobulins represent advantageously at least 85%, advantageously at least 86%, at least 87%, more advantageously at least 88%, at least 89%, even more advantageously at least 90%, at least 91%, at least 92%, at least 93%, at least 94%, even at least 95%, at least 96%, at least 97%, at least 98%, or at least 99% by weight of the total proteins of the composition.

The compositions according to the invention may comprise human polyclonal immunoglobulins of a single isotype (IgG, IgM, IgA, IgD, IgE, advantageously IgG or IgM, preferably IgG) or of several isotypes. However, in a preferred embodiment, the human polyclonal immunoglobulins present in the compositions according to the invention are mainly (at least 90%, advantageously at least 91%, at least 92%, at least 93%, at least 94%, more advantageously at least 95%, at least 96%, at least 97%, even more advantageously at least 98%, at least 99% by weight) IgG. In this case, the compositions according to the invention are advantageously enriched in subclass IgG2 immunoglobulins as compared to human normal immunoglobulin concentrates. In particular, IgG compositions according to the invention are advantageously characterized in that at least 40%, at least 45%, at least 50%, at least 55%, at least 60%, at least 65%, at least 70%, at least 75%, or even at least 80% by weight of the human polyclonal immunoglobulins of the IgG isotype that are present in the composition are subclass IgG2 immunoglobulins, advantageously measured by nephelometry and/or by spectrography and/or by ELISA kits for subclass determination (i.e., by nephelometry, by spectrography, by ELISA kits for subclass determination, or by several of these methods, for instance by nephelometry and by spectrography, or by each of these three methods). Alternatively or in addition, the compositions of IgG according to the invention advantageously have an IgG2:IgG1 weight ratio of at least 0.8, at least 0.9, advantageously at least 1, at least 1.1, at least 1.2, at least 1.3, at least 1.4, at least 1.5, at least 1.6, at least 1.7, at least 1.8, at least 1.9, or even at least 2, at least 2.5, at least 3, at least 3.1, at least 3.2, at least 3.3, at least 3.4, at least 3.5, at least 3.6, at least 3.7, at least 3.8, at least 3.9, or even at least 4, advantageously measured by nephelometry and/or by spectrography and/or by ELISA kits for subclass determination (i.e., by nephelometry, by spectrography, by ELISA kits for subclass determination, or by several of these methods, for instance by nephelometry and by spectrography, or by each of these three methods).

In another preferred embodiment, the human polyclonal immunoglobulins present in the compositions according to the invention are mainly (at least 90%, advantageously at least 91%, at least 92%, at least 93%, at least 94%, more advantageously at least 95%, at least 96%, at least 97%, even more advantageously at least 98%, at least 99% by weight) IgM.

In an advantageous embodiment, at least 80%, at least 81%, at least 82%, at least 83%, at least 84%, advantageously at least 85%, at least 86%, at least 87%, more advantageously at least 88%, at least 89%, even more advantageously at least 90%, at least 91%, at least 92%, at least 93%, at least 94%, or even at least 95%, at least 96%, at least 97%, at least 98%, or at least 99% by weight of human polyclonal immunoglobulins present in the composition are human anti-A polyclonal immunoglobulins, as defined above.

In another advantageous embodiment, at least 80%, at least 81%, at least 82%, at least 83%, at least 84%, advantageously at least 85%, at least 86%, at least 87%, more advantageously at least 88%, at least 89%, even more advantageously at least 90%, at least 91%, at least 92%, at least 93%, at least 94%, or even at least 95%, at least 96%, at least 97%, at least 98%, or at least 99% by weight of human polyclonal immunoglobulins present in the composition are human anti-B polyclonal immunoglobulins, as defined above.

In yet another advantageous embodiment, the composition comprises both human anti-A polyclonal immunoglobulins and human anti-B polyclonal immunoglobulins, and the weight ratio of human anti-A polyclonal immunoglobulins to human anti-B polyclonal immunoglobulins (anti-A:anti-B) is comprised between 1:10 and 10:1.

In an embodiment, the composition comprising both human anti-A polyclonal immunoglobulins and human anti-B polyclonal immunoglobulins is enriched in human anti-A polyclonal immunoglobulins, and thus has a weight ratio of human anti-A polyclonal immunoglobulins to human anti-B polyclonal immunoglobulins (anti-A:anti-B) comprised between 2:1 and 10:1.

In another embodiment, the composition comprising both human anti-A polyclonal immunoglobulins and human anti-B polyclonal immunoglobulins is enriched in human anti-B polyclonal immunoglobulins, and thus has a weight ratio of human anti-A polyclonal immunoglobulins to human anti-B polyclonal immunoglobulins (anti-A:anti-B) comprised between 1:10 and 1:2.

In another embodiment, the composition comprising both human anti-A polyclonal immunoglobulins and human anti-B polyclonal immunoglobulins is balanced in terms of the two types of immunoglobulins, and thus has a weight ratio of human anti-A polyclonal immunoglobulins to human anti-B polyclonal immunoglobulins (anti-A:anti-B) comprised between 3:10 and 7:10, advantageously between 4:10 and 6:10.

Alternatively or in combination with the weight percentages or ratios mentioned above, the composition enriched in human anti-A polyclonal immunoglobulins according to the invention may have an anti-A activity enriched by a factor of at least 4, advantageously at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, at least 300, at least 350, at least 400, at least 450, at least 500, at least 600, at least 700, at least 800, at least 900, at least 1000, at least 1500, at least 2000, at least 2500, at least 3000, at least 4000, at least 5000, at least 6000, at least 7000, at least 8000, at least 9000, or even at least 10000 as compared to a lyophilized normal human immunoglobulin reference or an EDQM reference.

Alternatively or in combination with the weight percentages or ratios mentioned above, the composition enriched in human anti-B polyclonal immunoglobulins according to the invention may have an anti-B activity enriched by a factor of at least 4, advantageously at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, at least 300, at least 350, at least 400, at least 450, at least 500, at least 600, at least 700, at least 800, at least 900, at least 1000, at least 1500, at least 2000, at least 2500, at least 3000, at least 4000, at least 5000, at least 6000, at least 7000, at least 8000, at least 9000, at least 10000, at least 11000, at least 12000, at least 13000, at least 14000, at least 15000, at least 16000, at least 17000, at least 18000, or even at least 19000 as compared to a lyophilized normal human immunoglobulin reference or to an EDQM reference.

Alternatively or in combination with the weight percentages or ratios mentioned above, the composition can comprise both human anti-A polyclonal immunoglobulins and human anti-B polyclonal immunoglobulins and has a ratio (anti-A activity:anti-B activity) comprised between 1:10 and 10:1, in particular between 1:9 and 9:1, between 1:8 and 8:1, between 1:7 and 7:1, between 1:6 and 6:1, between 1:5 and 5:1, between 1:4 and 4:1, between 1:3 and 3:1, or even between 1:2 and 2:1 or between 0.6 and 1.5.

The anti-A activity and the anti-B activity are determined by means of a method for assaying activity (such as one of those described below, and in particular the flow cytometry method described herein) and expressed in arbitrary units with respect to the same reference standard (EDQM reference standard Y0001688 or a lyophilized human normal immunoglobulin medicament).

The ratio (anti-A activity: anti-B activity) and the ratio (anti-B activity:anti-A activity) are calculated on the basis of anti-A and anti-B activity results obtained in tests carried out in parallel, with the same activity assay method (such as one of those described below, and in particular the flow cytometry method described herein) and expressed in arbitrary units with respect to the same reference standard (EDQM reference standard Y0001688 or a lyophilized human normal immunoglobulin medicament).

The compositions according to the invention are purified and are advantageously concentrated.

Advantageously, the compositions according to the invention are concentrated using any method known to a skilled person, for instance using an ultrafiltration membrane, a centrifugation, a dialysis, or several of these steps.

Even more advantageously, the compositions according to the invention have a concentration sufficient to permit successive dilutions of the composition for use as a standard range in a suitable assay method, for example the indirect Coombs test, the direct agglutination method, flow cytometry, or by several of these methods (for example, the indirect Coombs test and the direct agglutination method; the indirect Coombs test and flow cytometry; the direct agglutination method and flow cytometry; or each of these three methods).

Advantageously, the concentrated compositions according to the invention have a result in the indirect Coombs test (described below) superior to 1:64, advantageously superior to 1:128, superior to 1:256, superior to 1:512, superior to 1:1024, superior to 1:2048, or even superior to 1:4096, in order to permit successive dilutions of the composition to be used to prepare a standard range in the indirect Coombs test assay method. By result in the indirect Coombs test superior to 1:N is meant that the result of the indirect Coombs test is negative at a sample dilution of 1:N.

Alternatively or in addition, in an advantageous manner, the concentrated compositions according to the invention have a result in the direct agglutination method (described below) superior to 1:64, advantageously superior to 1:128, superior to 1:256, superior to 1:512, superior to 1:1024, superior to 1:2048, or even superior to 1:4096, in order to permit successive dilutions of the composition to be used to prepare a standard range in the direct agglutination method. By result in the direct agglutination test superior to 1:N is meant that the result in the direct agglutination method is negative at a sample dilution of 1:N.

Alternatively or in addition, in an advantageous manner, the concentrated compositions according to the invention have a result in the flow cytometry method (described below) superior to 1:64, advantageously superior to 1:128, superior to 1:256, superior to 1:512, superior to 1:1024, superior to 1:2048, or even superior to 1:4096, in order to permit successive dilutions of the composition to be used to prepare a standard range in the flow cytometry method. By result in the flow cytometry test superior to 1:N is meant that the result in the flow cytometry method is negative at a sample dilution of 1:N.

In an even more advantageous manner, the concentrated compositions according to the invention have a concentration in human anti-A and/or anti-B polyclonal immunoglobulins superior to 1 g/l, superior to 1.5 g/l, superior to 2 g/l, superior to 5 g/l, superior to 10 g/l, superior to 15 g/l, superior to 20 g/l, or even superior to 50 g/l.

Preparation of the Compositions According to the Invention

The compositions according to the invention may be obtained from varyingly purified fractions of human plasma comprising polyclonal immunoglobulins using various purification methods.

In a second aspect, the present invention thus relates to a method for preparing a composition according to the invention, comprising the following steps:
a) adsorbing a batch of human plasma or a human plasma fraction enriched in human polyclonal immunoglobulins on a support grafted with a specific ligand of human anti-A polyclonal immunoglobulins and/or with a specific ligand of human anti-B polyclonal immunoglobulins,
b) storing the unadsorbed fraction for possible later use, and
c) dissociating and collecting the adsorbed fraction.

In a particular embodiment, the present invention thus relates to a method for preparing a composition according to the invention, comprising the following steps:
a) adsorbing a batch of human plasma or a human plasma fraction enriched in human polyclonal immunoglobulins on a support grafted with a specific ligand of human anti-A polyclonal immunoglobulins,
b) storing the unadsorbed fraction for possible later use, and
c) dissociating and collecting the adsorbed fraction.

In another particular embodiment, the present invention thus relates to a method for preparing a composition according to the invention, comprising the following steps:
a) adsorbing a batch of human plasma or a human plasma fraction enriched in human polyclonal immunoglobulins on a support grafted with a specific ligand of human anti-B polyclonal immunoglobulins,
b) storing the unadsorbed fraction for possible later use, and
c) dissociating and collecting the adsorbed fraction.

Preparation of the compositions according to the invention is based on a step of specific adsorption of human anti-A and/or anti-B polyclonal immunoglobulins on a support grafted with a specific ligand of said immunoglobulins, which are then eluted.

Advantageously, it is possible to start directly from plasma or from a human plasma fraction enriched in human polyclonal immunoglobulins, varyingly purified for human polyclonal immunoglobulins.

In a particular embodiment, the method for preparing the compositions according to the invention may be integrated into a more general method for purifying therapeutic human normal immunoglobulins (collection of heretofore discarded fractions) and may thus be used on a pre-purified human polyclonal immunoglobulin fraction. Said pre-purified human polyclonal immunoglobulin fraction advantageously has a human polyclonal immunoglobulin content of at least 80%, advantageously at least 81%, at least 82%, more advantageously at least 83%, at least 84%, even more advantageously at least 85%, at least 86%, at least 87%, at least 88%, at least 89%, or even at least 90%, at least 91%, or at least 92% by weight of the total proteins of the fraction.

As indicated above, the pre-purified human polyclonal immunoglobulin fraction may in particular have been obtained by chromatographic separation, in particular according to the human polyclonal immunoglobulin purification methods described in WO99/64462 and WO02/092632, and more particularly in WO02/092632. In this case, the pre-purified human polyclonal immunoglobulin fraction is obtained via pre-purification by a step of precipitation of lipid contaminants of blood plasma or of an IgG-enriched blood plasma fraction, and a single step of chromatography on an anion-exchange resin carried out at alkaline pH, with selective elution of IgG in one step using a suitable buffer at a pH comprised between 4 to 7. Advantageously, pre-purification using a step of precipitation of lipid contaminants consists of a caprylic acid precipitation step.

When a pre-purified human polyclonal immunoglobulin fraction is used in step a) of the method described above, the pre-purified human polyclonal immunoglobulin fraction may also have undergone a biological safety step (virus removal and/or virus inactivation, in particular by solvent-detergent treatment), a concentration step (in particular by ultrafiltration), and/or a sterilizing filtration step.

In a preparation method according to the invention, the support may be any suitable support likely to be selected by the skilled person for adsorbing human anti-A and/or anti-B polyclonal immunoglobulins.

Such a support used in step a) is advantageously in the form of:
a) particles (in particular polymer particles) grafted with the ligand(s) of interest, or
b) a polymer membrane, the membrane being grafted with the ligand(s) of interest.

The support may thus in particular be in the form of particles grafted with the ligand(s) of interest. The particles are advantageously spherical or oblong in shape, and in particular may be beads. Said particles generally have a mean size of about 0.1 μm to about 1000 μm, preferably of about 20 μm to about 500 μm, more preferably of about 50 μm to about 200 μm, still more preferably of about 70 μm to about 120 μm. They may consist of polymer or of inorganic matter (such as silica or glass, for example). Advantageously, the particles are porous.

In an advantageous embodiment, they are polymer particles. The polymer may be natural or non-natural (synthetic or semisynthetic), organic or inorganic (preferably the polymer will be organic), cross-linked or not cross-linked (preferably the polymer will be cross-linked). Advantageously, the polymer is a cross-linked organic polymer.

The polymer may in particular be selected from cellulose and derivatives thereof, agarose, dextran, polyacrylates, polystyrene, polyacrylamide, polymethacrylamide, styrene and divinylbenzene copolymers, or mixtures of said polymers.

In a preferred embodiment, the polymer is cellulose, and the particles are preferably porous cellulose beads. More preferably still, it is cross-linked cellulose.

The support may also be in the form a polymer membrane, the membrane being grafted with the ligand(s) of interest. The membrane polymer may be selected from the polymers mentioned above for polymer particles.

The particles are advantageously incorporated into a gel or a resin, which is used as the matrix in an affinity chromatography column. In the same way, the polymer membrane may be included in an affinity chromatography column. The batch of human plasma or the human plasma fraction enriched in human polyclonal immunoglobulins is then adsorbed on the affinity chromatography column, and the adsorbed fraction is eluted and collected. However, although preferred, the use of an affinity chromatography column is not essential, and other methods of adsorption, dissociation and collection may be used.

In a preparation method according to the invention, the specific ligand of human anti-A polyclonal immunoglobulins may be any suitable molecule known to the skilled person that binds to human anti-A polyclonal immunoglobulins as defined above and that does not bind to other immunoglobulins.

Such a ligand is advantageously selected from oligosaccharides representative of type 1, 2, 3 and 4 group A antigens and in particular from the following oligosaccharides:
Trisaccharide: GalNAcα1-3(Fucα1-2)Gal;
Tetrasaccharides:
   Type 1: GalNAcα1-3(Fucα1-2)Galβ1-3GlcNAc,
   Type 2: GalNAcα1-3(Fucα1-2)Galβ1-4GlcNAc,
   Type 3 or 4: GalNAcα1-3(Fucα1-2)Galβ1-3GalNAc,
Pentasaccharides:
   Type 1: GalNAcα1-3(Fucα1-2)Galβ1-3GlcNAc β1-3Gal,
   Type 2: GalNAcα1-3(Fucα1-2)Galβ1-4GlcNAc β1-3Gal,
   Type 3: GalNAcα1-3(Fucα1-2)Galβ1-3GalNAc α1-3Gal,
   Type 4: GalNAcα1-3(Fucα1-2)Galβ1-3GalNAc β1-3Gal,
Hexasaccharides:
   Type 1: GalNAcα1-3(Fucα1-2)Galβ1-3GlcNAc β1-3Galβ1-4Glc,
   Type 2: GalNAcα1-3(Fucα1-2)Galβ1-4GlcNAc β1-3Galβ1-4Glc,
   Type 3: GalNAcα1-3(Fucα1-2)Galβ1-3GalNAc α1-3Galβ1-4GlcNAc,
   Type 4: GalNAcα1-3(Fucα1-2)Galβ1-3GalNAc β1-3Galα1-4Gal.

In a preparation method according to the invention, the specific ligand of human anti-B polyclonal immunoglobulins may be any suitable molecule known to the skilled person that binds to human anti-B polyclonal immunoglobulins as defined above and that does not bind to other immunoglobulins.

Such a ligand is advantageously selected from oligosaccharides representative of type 1, 2, 3 and 4 group B antigens and in particular from the following oligosaccharides:
Trisaccharide: Galα1-3(Fucα1-2)Gal;
Tetrasaccharides:
   Type 1: Galα1-3(Fucα1-2)Galβ1-3GlcNAc,
   Type 2: Galα1-3(Fucα1-2)Galβ1-4GlcNAc,
   Type 3 or 4: Galα1-3(Fucα1-2)Galβ1-3GalNAc,
Pentasaccharides:
   Type 1: Galα1-3(Fucα1-2)Galβ1-3GlcNAc β1-3Gal,
   Type 2: Galα1-3(Fucα1-2)Galβ1-4GlcNAc β1-3Gal,
   Type 3: Galα1-3(Fucα1-2)Galβ1-3GalNAc α1-3Gal,
   Type 4: Galα1-3(Fucα1-2)Galβ1-3GalNAc β1-3Gal,
Hexasaccharides:
   Type 1: Galα1-3(Fucα1-2)Galβ1-3GlcNAc β1-3Galβ1-4Glc,
   Type 2: Galα1-3(Fucα1-2)Galβ1-4GlcNAc β1-3Galβ1-4Glc,
   Type 3: Galα1-3(Fucα1-2)Galβ1-3GalNAc α1-3Galβ1-4GlcNAc,
   Type 4: Galα1-3(Fucα1-2)Galβ1-3GalNAc β1-3Galα1-4Gal.

In step a) of the method according to the invention as described above, the support may be grafted with a specific ligand of human anti-A polyclonal immunoglobulins and/or with a specific ligand of human anti-B polyclonal immunoglobulins.

In an embodiment, the support is grafted only with a specific ligand of human anti-A polyclonal immunoglobulins.

In another embodiment, the support is grafted only with a specific ligand of human anti-B polyclonal immunoglobulins.

In yet another embodiment, the support is grafted both with a specific ligand of human anti-A polyclonal immunoglobulins and with a specific ligand of human anti-B polyclonal immunoglobulins. In this case, a mixture of supports grafted with a specific ligand of human anti-A polyclonal immunoglobulins and of supports grafted with a specific ligand of human anti-B polyclonal immunoglobulins, in respective proportions generally comprised between 25:75 (v/v) and 75:25 (v/v), and in particular of 50:50 (v/v), will advantageously be used. In particular, when particles grafted with the ligand(s) of interest are used, a mixture of particles grafted with a specific ligand of human anti-A polyclonal immunoglobulins and of particles grafted with a specific ligand of human anti-B polyclonal immunoglobulins may be used to prepare a gel and to fill an affinity chromatography column. In this case, particles grafted with a specific ligand of human anti-A polyclonal immunoglobulins and particles grafted with a specific ligand of human anti-B polyclonal immunoglobulins are mixed in respective proportions generally comprised between 25:75 (v/v) and 75:25 (v/v), and in particular of 50:50 (v/v). In another embodiment, it is also possible to use a support comprising particles grafted with both a specific ligand of human anti-A polyclonal immunoglobulins and a specific ligand of human anti-B polyclonal immunoglobulins. In another embodiment, it is also possible to use a mixture of:
particles grafted with both a specific ligand of human anti-A polyclonal immunoglobulins and a specific ligand of human anti-B polyclonal immunoglobulins, and
particles grafted with a specific ligand of human anti-A polyclonal immunoglobulins and/or particles grafted with a specific ligand of human anti-B polyclonal immunoglobulins.

In a preparation method according to the invention, the ligand of interest is advantageously grafted onto the polymer particles or onto the polymer membrane via a spacer, which reduces steric hindrance and makes the trisaccharide characteristic of A or B antigens more accessible to immunoglobulins able to be adsorbed on the support.

Such a spacer may be any suitable group known to the skilled person that allows the ligand of interest, and thus oligosaccharides in particular, to be grafted onto a support of interest, in particular the polymers described above.

The spacer typically comprises at least one C, O, N, or S atom, and will generally comprise at least one of the following chemical functional groups: ether (—O—), thioether (—S—), amino (—NH—), carboxy -(—COO— or —OCO—), amide (—CONH— or —HNOC—).

It may in particular be selected from:
—(CH₂)mX(CH₂)n- or —(CH₂)mX1(CH₂)nX2(CH₂)p-, wherein X, X1, and X2 are each independently selected from O, S, NH and a covalent bond; m, n, and p are each independently 0, 1, 2, 3, 4, 5 or 6; and 1, 2 or 3 of the hydrogen atoms may be replaced by an equivalent number of OH and/or methyl groups.
The spacer may in particular have a structure selected from:

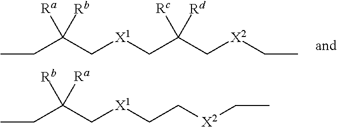

wherein each of X1 and X2 is independently selected from O, S, and NH; and each of Ra, Rb, Rc, and Rd is independently selected from H, OH, and methyl.
One of the following structures:

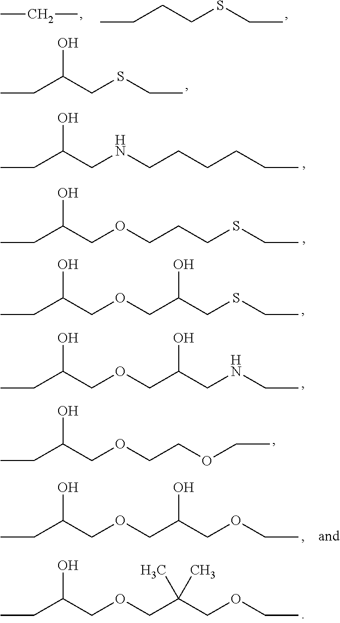

Spacers of formula —NH—R1—CONH—R2—, wherein R1 is a C4-C6 alkyl group, R2 is a C3-C8 alkyl group, and said spacer is linked by its amine functional group (in bold above) to the support.
In this case, R1 is a linear or branched, preferably linear C4-C6 alkyl group. Preferably, R1 is a C5 alkyl group.
R2 is a linear or branched, preferably linear C3-C8 alkyl group. Preferably, R2 is a C3 alkyl group.
In a preferred embodiment, the ligands (which are preferably trisaccharides as described above) are grafted onto the particles or the membrane via a spacer of formula: (particle/membrane)—NH—C₅H₁₀—CO—NH—C₃H6—(ligand).

Coupling between the particle or the membrane and the spacer, on the one hand, and coupling between the spacer and the specific ligand of human anti-A polyclonal immunoglobulins or the specific ligand of human anti-B polyclonal immunoglobulins, on the other, may be carried out by any suitable chemical synthesis protocol known to the skilled person.

In a particular embodiment, the particle or the membrane may carry an —NH—R1—COOH arm. Preferably, said arm is ϵ-aminocaproic acid (wherein R1 is a pentyl group). Conventionally, the particle may then be activated using bifunctional reagents such as epichlorohydrin, epibromohydrin, dibromo- and dichloropropanol, dibromobutane, ethylene glycol diglycidyl ether, butanediol diglycidyl ether, divinyl sulfone, allyl glycidyl ether, and allyl bromide. The bifunctional reagent is able to react with both the particles/membrane and the —NH—R1—COOH arm. Heterofunctional allylic compounds, such as allyl bromide, are preferred bifunctional reagents and make it possible to produce an activated matrix.

For certain solid supports, such as cellulose, composites containing hydrogel, or other materials having hydroxyl groups, it is advantageous to deprotonate the hydroxyl groups with a hydroxide source, for example, before reaction with a bifunctional reagent.

The ligands representing antigens of blood groups A and/or B are then immobilized on the activated particle/membrane carrying the —NH—R1—COOH arm via an —NH—R2— linking group, wherein R2 is a linear or branched, preferably linear C3-C8 alkyl group. To that end, the COOH functional group of the —NH—R1—COOH arm carried by the particle/membrane is reacted with the NH₂ functional group of the NH₂—R2— oligosaccharide ligand, by use of an N-ethoxycarbonyl-2-ethoxy-1,2-dihydroquinoline (EEDQ)-type condensation agent.

Examples of supports grafted with a specific ligand of human anti-A polyclonal immunoglobulins that may be used in the context of the invention are as follows: Glycosorb ABO A (Sepharose matrix to which the trisaccharide characteristic of A antigen is grafted, Glycorex Transplantation AB, Lund, Sweden), Allotran A (trisaccharide characteristic of A antigen grafted onto a Sepharose FF matrix via polyacrylamide, Lectinity Corp), HyperCel IsoA (cross-linked cellulose particles grafted with the trisaccharide characteristic of A antigen, Pall).

Examples of supports grafted with a specific ligand of human anti-B polyclonal immunoglobulins that may be used in the context of the invention are as follows: Glycosorb ABO B (Sepharose matrix to which the trisaccharide characteristic of B antigen is grafted, Glycorex Transplantation AB, Lund, Sweden), Allotran B (trisaccharide characteristic of B antigen grafted onto a Sepharose FF matrix via polyacrylamide, Lectinity Corp), HyperCel IsoB (cross-linked cellulose particles grafted with the trisaccharide characteristic of B antigen, Pall).

When one wishes to use a support grafted with a specific ligand of human anti-A polyclonal immunoglobulins and a specific ligand of human anti-B polyclonal immunoglobulins, a mixture of a support grafted with a specific ligand of human anti-A polyclonal immunoglobulins as described above and a support grafted with a specific ligand of human anti-B polyclonal immunoglobulins as described above will generally be used. In particular, when particles grafted with the ligand(s) of interest are used, a mixture of particles grafted with a specific ligand of human anti-A polyclonal immunoglobulins and of particles grafted with a specific ligand of human anti-B polyclonal immunoglobulins may be used to prepare a gel and to fill an affinity chromatography column. Particles grafted with both a specific ligand of human anti-A polyclonal immunoglobulins and a specific ligand of human anti-B polyclonal immunoglobulins may also be used to prepare a gel and to fill an affinity chromatography column. In another embodiment, it is also possible to use a mixture of:
particles grafted with both a specific ligand of human anti-A polyclonal immunoglobulins and a specific ligand of human anti-B polyclonal immunoglobulins, and
particles grafted with a specific ligand of human anti-A polyclonal immunoglobulins and/or particles grafted with a specific ligand of human anti-B polyclonal immunoglobulins.

When purification of the composition according to the invention is carried out by affinity chromatography, the batch of human plasma or the human plasma fraction enriched in human polyclonal immunoglobulins is adsorbed in step a) on the chromatography column under any suitable condition known to the skilled person, in particular any condition recommended by the manufacturer of the chromatography support, depending on the support selected. In particular, the batch of human plasma or the human plasma fraction enriched in human polyclonal immunoglobulins may be percolated on the column. The unadsorbed fraction is advantageously recovered for other later uses. The adsorbed fraction is then dissociated and collected using one or more washes of the column with one or more suitable elution buffers. In particular, acidic elution buffer (glycine-HCl buffer, pH 2 to 4, for example) and/or basic elution buffer (glycine-NaOH solution, pH 10 to 12, for example) may be used.

Furthermore, the composition thus obtained may undergo one or more subsequent optional steps, such as: a step of neutralization of the composition (adjustment of pH between 3 and 9, preferably between 4 and 5), one or more additional purification steps, a concentration step (by ultrafiltration, for example), at least one inactivation step (solvent-detergent treatment, for example) or virus removal step (nanofiltration, for example), or a combination of several of these steps.

The method according to the invention as described above enables the obtention of a composition of human polyclonal immunoglobulins of which at least 80%, at least 81%, at least 82%, at least 83%, at least 84%, advantageously at least 85%, at least 86%, at least 87%, more advantageously at least 88%, at least 89%, even more advantageously at least 90%, at least 91%, at least 92%, at least 93%, at least 94%, or even at least 95%, at least 96%, at least 97%, at least 98%, or at least 99% by weight of the polyclonal human immunoglobulins present in the composition recognize antigens of blood groups A and B. When the support used in step a) is grafted only with a specific ligand of human anti-A polyclonal immunoglobulins, human anti-A polyclonal immunoglobulins are retained, and the obtained composition thus comprises human anti-A polyclonal immunoglobulins.

Alternatively, the support used in step a) is grafted only with a specific ligand of human anti-B polyclonal immunoglobulins, human anti-B polyclonal immunoglobulins are retained, and the obtained composition thus comprises human anti-B polyclonal immunoglobulins.

When the support used in step a) is grafted both with a specific ligand of human anti-A polyclonal immunoglobulins and with a specific ligand of human anti-B polyclonal immunoglobulins, the purified composition then comprises a mixture of human anti-A polyclonal immunoglobulins and of human anti-B polyclonal immunoglobulins. In human polyclonal immunoglobulins purified from plasma pools, the proportion of human anti-A polyclonal immunoglobulins and human anti-B polyclonal immunoglobulins depends on the initial donor population. Indeed, different donor populations have different blood group distributions. These differences may thus be found in the compositions of human anti-A polyclonal immunoglobulins and/or of human anti-B polyclonal immunoglobulins according to the invention.

In order to obtain either a purified composition of human anti-A polyclonal immunoglobulins or a purified composition of human anti-B polyclonal immunoglobulins, the inventors have developed novel purification methods.

Thus, in order to prepare a composition according to the invention wherein at least 80%, at least 81%, at least 82%, at least 83%, at least 84%, advantageously at least 85%, at least 86%, at least 87%, more advantageously at least 88%, at least 89%, even more advantageously at least 90%, at least 91%, at least 92%, at least 93%, at least 94%, or even at least 95%, at least 96%, at least 97%, at least 98%, or at least 99% by weight of human polyclonal immunoglobulins present in the composition are human anti-A polyclonal immunoglobulins, the method advantageously comprises the following steps:
a) adsorbing a batch of human plasma or a human plasma fraction enriched in human polyclonal immunoglobulins on a support grafted with a specific ligand of human anti-A polyclonal immunoglobulins, and with a specific ligand of human anti-B polyclonal immunoglobulins,
b) storing the unadsorbed fraction for possible later use,
c) dissociating and collecting the adsorbed fraction,
d) adsorbing the composition obtained in step c) on a support grafted with a specific ligand of human anti-B polyclonal immunoglobulins, and
e) collecting the unadsorbed fraction.

Steps a) to c) are identical to those described above for preparing a composition according to the invention wherein at least 80% by weight of human polyclonal immunoglobulins present in the composition are human anti-A and/or anti-B polyclonal immunoglobulins. In particular, the conditions for adsorbing, storing the unadsorbed fraction and dissociating and collecting the adsorbed fraction may be similar to those described above. Steps d) and e) aim to specifically isolate human anti-A polyclonal immunoglobulins. To this end, in step d) a support grafted with a specific ligand of human anti-B polyclonal immunoglobulins is used such that all the human anti-B polyclonal immunoglobulins are adsorbed on the support. Human anti-A polyclonal immunoglobulins may thus be collected directly (step e)), producing a composition specifically enriched in human anti-A polyclonal immunoglobulins. Advantageously, the conditions in step d) are adapted to enable the adsorption of all of the human anti-B polyclonal immunoglobulins on the support, for example by adapting gel volume and/or residence time. When the support used in step d) is an affinity chromatography column that uses a membrane or a gel of particles grafted with a specific ligand of human anti-B polyclonal immunoglobulins as a support, the adsorption conditions may be similar to those described previously for step a). The unadsorbed fraction is then collected directly in step e).

In another particular embodiment of the invention aiming to prepare a composition according to the invention in which at least 80%, at least 81%, at least 82%, at least 83%, at least 84%, advantageously at least 85% at least 86%, at least 87%, more advantageously at least 88%, at least 89%, even more advantageously at least 90%, at least 91%, at least 92% at least 93%, at least 94%, or even at least 95%, at least 96%, at least 97%, at least 98%, or at least 99% by weight of human polyclonal immunoglobulins present in the composition are human anti-A polyclonal immunoglobulins, the method advantageously comprises the following steps:
a) adsorbing a batch of human plasma or a human plasma fraction enriched in human polyclonal immunoglobulins on a support grafted with a specific ligand of human anti-A polyclonal immunoglobulins, and with a specific ligand of human anti-B polyclonal immunoglobulins,
b) storing the unadsorbed fraction for possible later use,
c) dissociating and collecting the adsorbed fraction,
d) adsorbing the composition obtained in step c) on a support grafted with a specific ligand of human anti-A polyclonal immunoglobulins, and
e) dissociating and collecting the adsorbed fraction.

Alternatively, in order to prepare a composition according to the invention wherein at least 80%, at least 81%, at least 82%, at least 83%, at least 84%, advantageously at least 85%, at least 86%, at least 87%, more advantageously at least 88%, at least 89%, even more advantageously at least 90%, at least 91%, at least 92%, at least 93%, at least 94%, or even at least 95%, at least 96%, at least 97%, at least 98%, or at least 99% by weight of human polyclonal immunoglobulins present in the composition are human anti-B polyclonal immunoglobulins, the method advantageously comprises the following steps:
a) adsorbing a batch of human plasma or a human plasma fraction enriched in human polyclonal immunoglobulins on a support grafted with a specific ligand of human anti-B polyclonal immunoglobulins, and with a specific ligand of human anti-A polyclonal immunoglobulins,
b) storing the unadsorbed fraction for possible later use,
c) dissociating and collecting the adsorbed fraction,
d) adsorbing the composition obtained in step c) on a support grafted with a specific ligand of immunoglobulins that recognize blood group A antigens, and
e) collecting the unadsorbed fraction.

Steps a) to c) are identical to those described above for the preparation of a composition according to the invention wherein at least 80% by weight of human polyclonal immunoglobulins present in the composition are human anti-A or anti-B polyclonal immunoglobulins. In particular, the conditions for adsorbing, storing the unadsorbed fraction and dissociating and collecting the adsorbed fraction may be similar to those described above.

Steps d) and e) aim to specifically isolate human anti-B polyclonal immunoglobulins. To this end, in step d) a support grafted with a specific ligand of human anti-A polyclonal immunoglobulins is used such that all the human anti-A polyclonal immunoglobulins are adsorbed on the support. Human anti-B polyclonal immunoglobulins may thus be collected directly (step e)), producing a composition specifically enriched in human anti-B polyclonal immunoglobulins. Advantageously, the conditions in step d) are adapted to enable the adsorption of all of the human anti-B polyclonal immunoglobulins on the support, for example by adapting gel volume and/or residence time. When the support used in step d) is an affinity chromatography column that uses a membrane or a gel of particles grafted with a specific ligand of human anti-A polyclonal immunoglobulins as a support, the adsorption conditions may be similar to those described previously for step a). The unadsorbed fraction is then collected directly in step e).

In another particular embodiment of the invention aiming to prepare a composition according to the invention in which at least 80%, at least 81%, at least 82%, at least 83%, at least 84%, advantageously at least 85% at least 86%, at least 87%, more advantageously at least 88%, at least 89%, even more advantageously at least 90%, at least 91%, at least 92% at least 93%, at least 94%, or even at least 95%, at least 96%, at least 97%, at least 98%, or at least 99% by weight of human polyclonal immunoglobulins present in the composition are human anti-B polyclonal immunoglobulins, the method advantageously comprises the following steps:
a) adsorbing a batch of human plasma or a human plasma fraction enriched in human polyclonal immunoglobulins on a support grafted with a specific ligand of human anti-A polyclonal immunoglobulins, and with a specific ligand of human anti-B polyclonal immunoglobulins,
b) storing the unadsorbed fraction for possible later use,
c) dissociating and collecting the adsorbed fraction,
d) adsorbing the composition obtained in step c) on a support grafted with a specific ligand of human anti-B polyclonal immunoglobulins, and
e) dissociating and collecting the adsorbed fraction.

In a particular embodiment of the invention, the batch of human plasma or the human plasma fraction enriched in human polyclonal immunoglobulins (pre-purified fraction of human polyclonal immunoglobulins) unadsorbed in step a) is used in a method for preparing human polyvalent immunoglobulins. This particular embodiment of the invention advantageously enables the concomitant preparation:
of a concentrate of human polyvalent immunoglobulins, depleted of anti-A and anti-B,
of a composition specifically enriched in human anti-A polyclonal immunoglobulins, and
of a composition specifically enriched in human anti-B polyclonal immunoglobulins.

In an advantageous embodiment of the invention, the method for preparing a purified anti-A immunoglobulin composition thus comprises the following steps:
a) adsorbing a pre-purified fraction of human polyclonal immunoglobulins on a support grafted with a specific ligand of human anti-B polyclonal immunoglobulins, and with a specific ligand of human anti-A polyclonal immunoglobulins,
b) collecting of the unadsorbed fraction for use in a method of purifying a concentrate of human polyvalent immunoglobulins depleted of human anti-A and anti-B polyclonal immunoglobulins,
c) dissociating and collecting the fraction adsorbed in step a),
d) adsorbing the composition obtained in step c) on a support grafted with a specific ligand of immunoglobulins recognizing blood group B antigens, and
e) collecting the unadsorbed fraction.

In an advantageous embodiment of the invention, the method for preparing a purified anti-A immunoglobulin composition thus comprises the following steps:
a) adsorbing a pre-purified fraction of human polyclonal immunoglobulins, in particular a fraction of human plasma enriched in human polyclonal immunoglobulins obtained by anion exchange chromatography at alkaline pH, on a support grafted with a specific ligand of human anti-B polyclonal immunoglobulins, and with a specific ligand of human anti-A polyclonal immunoglobulins,
b) collecting of the unadsorbed fraction for use in a method of purifying a concentrate of human polyvalent immunoglobulins depleted of human anti-A and anti-B polyclonal immunoglobulins,
c) dissociating and collecting the fraction adsorbed in step a),
d) adsorbing the composition obtained in step c) on a support grafted with a specific ligand of immunoglobulins recognizing blood group B antigens, and
e) collecting the unadsorbed fraction.

In an advantageous embodiment of the invention, the method for preparing a purified anti-B immunoglobulin composition thus comprises the following steps:
a) adsorbing a pre-purified fraction of human polyclonal immunoglobulins on a support grafted with a specific ligand of human anti-B polyclonal immunoglobulins, and with a specific ligand of human anti-A polyclonal immunoglobulins,
b) collecting of the unadsorbed fraction for use in a method of purifying a concentrate of human polyvalent immunoglobulins depleted of human anti-A and anti-B polyclonal immunoglobulins,
c) dissociating and collecting the fraction adsorbed in step a),
d) adsorbing the composition obtained in step c) on a support grafted with a specific ligand of immunoglobulins recognizing blood group A antigens, and
e) collecting the unadsorbed fraction.

In an advantageous embodiment of the invention, the method for preparing a purified anti-B immunoglobulin composition thus comprises the following steps:
a) adsorbing a pre-purified fraction of human polyclonal immunoglobulins, in particular a fraction of human plasma enriched in human polyclonal immunoglobulins obtained by anion exchange chromatography at alkaline pH, on a support grafted with a specific ligand of human anti-B polyclonal immunoglobulins, and with a specific ligand of human anti-A polyclonal immunoglobulins,
b) collecting the unadsorbed fraction for use in a method of purifying a concentrate of human polyvalent immunoglobulins depleted of human anti-A and anti-B polyclonal immunoglobulins,
c) dissociating and collecting the fraction adsorbed in step a),
d) adsorbing the composition obtained in step c) on a support grafted with a specific ligand of immunoglobulins recognizing blood group A antigens, and
e) collecting the unadsorbed fraction.

In another advantageous embodiment of the invention, the method for preparing a purified anti-A immunoglobulin composition and a purified anti-B immunoglobulin composition thus comprises the following steps:
a) adsorbing a pre-purified fraction of human polyclonal immunoglobulins on a support grafted with a specific ligand of human anti-B polyclonal immunoglobulins, and with a specific ligand of human anti-A polyclonal immunoglobulins,
b) collecting the unadsorbed fraction for use in a method of purifying a concentrate of human polyvalent immunoglobulins depleted of human anti-A and anti-B polyclonal immunoglobulins,
c) dissociating and collecting the fraction adsorbed in step a),
d) adsorbing a part of the composition obtained in step c) on a support grafted with a specific ligand of immunoglobulins recognizing blood group B antigens, and collecting the unadsorbed fraction enriched in anti-A.
e) adsorbing the other part of the composition obtained in step c) on a support grafted with a specific ligand of immunoglobulins recognizing blood group A antigens, and collecting the unadsorbed fraction enriched in anti-B.

This particularly advantageous embodiment makes it possible optimize recovery of the retained and non-retained fractions from affinity chromatography using specific ligands of human anti-B polyclonal immunoglobulins and specific ligands of human anti-A polyclonal immunoglobulins.

In the methods for preparing compositions enriched specifically in anti-A immunoglobulins or in anti-B immunoglobulins described above, the supports and ligands used in steps a) and d) and/or e) may be selected from those described above for the general method for preparing a composition enriched in anti-A and anti-B immunoglobulins.

The invention further relates to a composition able to be obtained by one of the preparation methods described above.

Positive Controls Intended to be Used in a Method for Assaying Anti-A and/or Anti-B Activity of a Composition Comprising Human Polyclonal Immunoglobulins

The compositions according to the invention are useful in particular for preparing a positive control (below-limit positive control, limit positive control, or above-limit positive control) useful in a method for assaying anti-A and/or anti-B activity of human normal immunoglobulin concentrate, which does not have the disadvantages of the positive control and/or of the limit positive control available to date (fractions 07/306 and 07/310), i.e., which comprises human and polyclonal, and not murine and monoclonal, anti-A and/or anti-B immunoglobulins, and which can be prepared in sufficient amounts.

Thus, in a third aspect, the present invention relates to the use of a composition according to the invention as positive control in a method for assaying anti-A and/or anti-B activity of a composition comprising human polyclonal immunoglobulins.

The invention also relates to the use of a composition according to the invention for preparing a positive control (below-limit positive control, limit positive control, or above-limit positive control) intended for use in a method for assaying anti-A and/or anti-B activity of a composition comprising human polyclonal immunoglobulins.

The invention also relates to a method for preparing a positive control intended for use in a method for assaying anti-A and/or anti-B activity of a composition comprising human polyclonal immunoglobulins, comprising the following steps:
a) providing a composition comprising human polyclonal immunoglobulins having anti-A activity and anti-B activity inferior to a given limit value in said assay method, and
b) adding a composition according to the invention.

The invention also relates to a method for preparing a positive control (below-limit positive control, limit positive control, or above-limit positive control) intended for use in a method for assaying anti-A and/or anti-B activity of a composition comprising human polyclonal immunoglobulins, comprising the following steps:
a) providing a composition comprising human polyclonal immunoglobulins having anti-A activity and anti-B activity inferior to a given limit value in said assay method,
b) adding increasing amounts of a composition according to the invention in which at least 80% by weight of human polyclonal immunoglobulins present in the composition according to the invention are human anti-A polyclonal immunoglobulins and/or of a composition according to the invention in which at least 80% by weight of human polyclonal immunoglobulins present in the composition according to the invention are human anti-B polyclonal immunoglobulins, in order to obtain various compositions enriched in human anti-A and/or anti-B polyclonal immunoglobulins,
c) assaying by said assay method the anti-A and/or anti-B activity of compositions enriched in human anti-A and/or anti-B polyclonal immunoglobulins obtained in step b), and
d) selecting the composition enriched in human anti-A and/or anti-B polyclonal immunoglobulins having anti-A and/or anti-B activity in said assay method inferior to (in order to obtain a below-limit positive control), equal to (in order to obtain a limit positive control), or superior to (in order to obtain an above-limit positive control) said limit value.

A positive control (below-limit positive control, limit positive control, or above-limit positive control) may be prepared by the method above for any method for assaying anti-A and/or anti-B activity of interest, and for any limit value of interest.

In particular, a positive control (below-limit positive control, limit positive control, or above-limit positive control) may be prepared by the method above for one or other of the methods for assaying anti-A and/or anti-B activity generally used:
(i) a method called the indirect antiglobulin test (IAT, also called indirect Coombs test), which is based on the use of antiglobulin (i.e., immunoglobulins of animal origin that specifically recognize human immunoglobulins) to reveal human anti-A or anti-B polyclonal immunoglobulins bound to red blood cells of blood group A or B.
Detection of red blood cell agglutination in the presence of human anti-A and/or anti-B polyclonal immunoglobulins can be carried out by various methods of varying precision.
In the common IAT test, agglutination is read visually, by detection of the presence of a precipitate.
The IAT test is the test previously recommended by health authorities for assaying anti-A and/or anti-B activity of therapeutic human normal immunoglobulin concentrates.
(ii) a method called the direct agglutination method, developed by Thorpe and colleagues (Thorpe et al. Vox Blood. 2009 August; 97(2):160-8; Thorpe et al. Pharmeur Bio Sci Notes. 2010 April; 2010(1):39-50; document WHO/BS/08.2091, which can be downloaded from the address http://apps.who.int/iris/handle/10665/69970?locale=fr).
In this method, A or B red blood cells treated with papain are brought into contact with two-fold serial dilutions of a 5% (50 g/l) human normal immunoglobulin solution, in a microplate with V-shaped wells. After centrifugation, the plate is tilted at an angle of about 70° for about 4-5 minutes. If agglutination occurs, the cell pellet remains stuck to the bottom of the V-shaped well, forming a well-rounded point. Otherwise, the non-agglutinated cell pellet slides along the wall of the V-shaped well, thus generating a droplet-type shape.
The direct agglutination method is the new test recommended by health authorities for assaying anti-A and/or anti-B activity of therapeutic human normal immunoglobulin concentrates.
(iii) A cytometry test uses a F(ab′)₂ labeled with a fluorescent label. After adding labeled F(ab′)2 and washing unbound F(ab′)2, mean fluorescence intensity in the sample is measured and compared with that of one or more reference samples. A more precise description of this test is described in application WO2007/077365.

The limit value for which the limit positive control is prepared may be any anti-A and/or anti-B activity limit value of interest. In particular, it may be the maximum anti-A and/or anti-B activity value accepted by health authorities in a country or a region of the world. Currently, according to criteria of the European Pharmacopoeia, the WHO and the FDA, therapeutic human normal immunoglobulin concentrates should not show A and B red blood cell agglutination at a 1:64 dilution of a solution whose initial concentration is 50 g/l (5%), in a specific direct agglutination test corresponding to that developed by Thorpe and colleagues (European Pharmacopoeia, chapter 2.6.20), and this limit value may thus be selected in an advantageous manner. However, it should be noted that this limit value is likely to be changed by health authorities based on monitoring of future hemolysis accidents occurring with therapeutic concentrates that respect this limit value. The method described above could then be used again to prepare a new limit positive control, adapted to the new limit set by health authorities.

Moreover, other positive controls may be of use to the skilled person. For example, a range of below-limit positive controls with increasing anti-A and/or anti-B activities but that are inferior to the limit value set by health authorities could make it possible to determine whether a therapeutic concentrate is close to the maximum limit accepted by health authorities or significantly below this limit. In the same way, a range of above-limit positive controls with increasing anti-A activities and/or anti-B that are all superior to the limit value set by health authorities could make it possible to determine whether a therapeutic concentrate is close to the maximum limit accepted by health authorities or significantly above this limit.

In step a) of the method above, a composition comprising human polyclonal immunoglobulins having anti-A activity and anti-B activity inferior to a given limit value in said assay method (starting composition) is provided, to which increasing amounts of a composition according to the invention in which at least 80% by weight of human polyclonal immunoglobulins present in the composition according to the invention are human anti-A polyclonal immunoglobulins and/or of a composition according to the invention in which at least 80% by weight of human polyclonal immunoglobulins present in the composition according to the invention are human anti-B polyclonal immunoglobulins are added in step b) in order to obtain various compositions enriched in human anti-A and/or anti-B polyclonal immunoglobulins.

The starting composition may lack human anti-A and/or anti-B polyclonal immunoglobulins (for example by suitable selection of the plasma donors used), or may comprise human anti-A and/or anti-B polyclonal immunoglobulins, provided that its anti-A activity and its anti-B activity are inferior to the selected limit value in the selected assay method. When the selected limit value and assay method correspond to those stipulated by regulatory authorities, such a starting composition may in particular be a human polyclonal immunoglobulin composition that is purified and depleted of human anti-A and anti-B polyclonal immunoglobulins, obtained for example as described in WO2007/077365. The presence of human polyclonal immunoglobulins that do not recognize A and B antigens is intended to maintain the same total immunoglobulin concentration in the limit positive control as in the composition whose anti-A or anti-B activity will later be tested by the assay method. In particular, this starting composition has an immunoglobulin concentration preferably equal to or superior to that recommended in the anti-A and anti-B activity test required by health authorities (to date 50 g/l, or 5%).

In step b), increasing amounts of a composition according to the invention in which at least 80% by weight of human polyclonal immunoglobulins present in the composition according to the invention are human anti-A polyclonal immunoglobulins and/or of a composition according to the invention in which at least 80% by weight of human polyclonal immunoglobulins present in the composition according to the invention are human anti-B polyclonal immunoglobulins are added in order to obtain various compositions enriched to a greater or lesser degree in human anti-A and/or anti-B polyclonal immunoglobulins.

In step c), the anti-A and/or anti-B activity of compositions enriched in human anti-A and/or anti-B polyclonal immunoglobulins obtained in step b) is tested by the initially-selected assay method.

Finally, in step d), one selects the composition enriched in human anti-A and/or anti-B polyclonal immunoglobulins having anti-A and/or anti-B activity in said assay method inferior to (in order to obtain a below-limit positive control), equal to (in order to obtain a limit positive control), or superior to (in order to obtain an above-limit positive control) the initially-selected limit value.

If one seeks to prepare an above-limit positive control, and none of the compositions tested has anti-A and/or anti-B activity superior to the initially-selected limit value, steps a) to c) may be repeated until the composition having anti-A and/or anti-B activity superior to the initially-selected limit value is obtained.

Conversely, if one seeks to prepare a below-limit positive control, and none of the compositions tested has anti-A and/or anti-B activity inferior to the initially-selected limit value, steps a) to c) may be repeated until the composition having anti-A and/or anti-B activity inferior to the initially-selected limit value is obtained.

Finally, if one seeks to prepare a limit positive control, and none of the compositions tested has anti-A and/or anti-B activity equal to the initially-selected limit value, steps a) to c) may be repeated until the composition having anti-A and/or anti-B activity equal to the initially-selected limit value is obtained.

In particular, depending on whether the compositions tested in step c) have anti-A and/or anti-B activity inferior to, equal to, or superior to the initially-selected limit value, the skilled person will be able to adapt the amounts of the composition according to the invention to be added in step b) so as to be able to select the positive control of interest accordingly.

The invention further relates to a positive control (below-limit positive control, limit positive control, or above-limit positive control) intended for use in a method for assaying anti-A and/or anti-B activity of a composition comprising human polyclonal immunoglobulins, able to be obtained by the method according to the invention for preparing a positive control (below-limit positive control, limit positive control, or above-limit positive control) described above.

Determining Blood Type

The compositions according to the invention wherein at least 80% by weight of human polyclonal immunoglobulins present in the composition are human anti-A polyclonal immunoglobulins may in addition be used in a test for determining an individual's blood type.

In the same way, the compositions according to the invention wherein at least 80% by weight of human polyclonal immunoglobulins present in the composition are human anti-B polyclonal immunoglobulins may in addition be used in a test for determining an individual's blood type.

The compositions according to the invention are then brought into contact with red blood cells of an individual whose blood type one seeks to determine, and the blood type is determined in the following manner:
agglutination by the composition comprising at least 80% human anti-A polyclonal immunoglobulins, but not by the composition comprising at least 80% human anti-B polyclonal immunoglobulins: the patient is type A,
agglutination by the composition comprising at least 80% human anti-B polyclonal immunoglobulins but not by the composition comprising at least 80% human anti-A polyclonal immunoglobulins: the patient is type B,
agglutination by the composition comprising at least 80% human anti-A polyclonal immunoglobulins and by the composition comprising at least 80% human anti-B polyclonal immunoglobulins: the patient is type AB,
no agglutination by either of the two compositions: the patient is type O.

This method for determining blood types using compositions according to the invention will be advantageously supplemented by a search for specific antibodies in the patient's serum.

The following examples aim to illustrate the present invention.

### EXAMPLES

### Example 1

### Preparation of a Human Polyclonal Immunoglobulin Composition Enriched in Human Anti-A and Anti-B Polyclonal Immunoglobulins

A first human polyclonal immunoglobulin composition according to the invention enriched in human anti-A and anti-B polyclonal immunoglobulins was prepared.

Materials and Methods

A purified human polyclonal immunoglobulin composition was prepared from a plasma pool according to the method described in application WO02/092632.

Said purified human polyclonal immunoglobulin composition was then adsorbed on a 1 ml affinity chromatography column filled with gel comprising a mixture of porous cross-linked cellulose beads grafted with the trisaccharide characteristic of group A antigens (column A) and of porous cross-linked cellulose beads grafted with the trisaccharide characteristic of group B antigens (column B), in respective proportions of 50:50. The load was 1.8 kg of purified human polyclonal immunoglobulin composition per liter of gel. Contact time was set to 2 minutes.

The unadsorbed fraction is collected for subsequent processing in order to prepare a therapeutic human polyclonal immunoglobulin concentrate lacking human anti-A and anti-B polyclonal immunoglobulins.

The fraction of interest in the context of the present invention is then obtained by combining two elution fractions:
A first elution of the chromatography column with acidic buffer (0.1 M glycine, pH 3),
A second elution of the chromatography column with basic buffer (0.1 M glycine, pH 11),
   followed by neutralization of the combined fraction (pH adjusted to 7).

Said composition was then analyzed using standard technologies in order to determine the concentrations of IgG, IgA and IgM and the levels of polymers, dimers, monomers and immunoglobulin fragments.

The anti-A and anti-B activity of the composition was also analyzed by the method described in WO2007/077365 and compared with that of a lyophilized human normal immunoglobulin.

Results

The starting purified human polyclonal immunoglobulin composition, which was adsorbed on the anti-A and anti-B affinity chromatography column, has the following features:
Total proteins: 10.0 g/l
IgG: 9.20 g/l (Subclasses: IgG1: 65%; IgG2: 30%; IgG3: 3%; IgG4: 2%)
IgA: <0.013 g/l
IgM: <0.009 g/l
Molecular size distribution (MSD):
   Polymers: <0.4%
   Dimers: 5.4%
   Monomers: 93.5%
   Fragments: <1.0%

Following the anti-A and anti-B affinity chromatography step, the combined fraction of the two successive elutions with acidic buffer and then with basic buffer has the following features:
IgG: 0.34 g/l
IgA: 0.015 g/l
IgM: <6.3 mg/l
Anti-A: 622.3 AU*
Anti-B: 638.7 AU*
Molecular size distribution (MSD):
   Polymers: 0.1%
   Dimers: 2.9%
   Monomers: 95.6%
   Fragments: 1.3%

At this stage, anti-A activity and anti-B activity are expressed in arbitrary units (AU) with respect to a lyophilized human normal immunoglobulin, the reference product whose value is set to 1. The lyophilized human normal immunoglobulin, is a human normal immunoglobulin product granted a Marketing Authorization in France. The lyophilized human normal immunoglobulin thus has for anti-A and anti-B a negative result in the direct Coombs test at a 1:64 dilution as required by regulatory authorities. Thus, the composition obtained by the method according to the invention has anti-A activity and anti-B activity that is about 600 times superior to that of the human polyvalent normal immunoglobulins of the lyophilized human normal immunoglobulin (therapeutic human polyclonal immunoglobulin concentrate).

Conclusions

Results presented above show that it is possible to obtain a purified human anti-A and anti-B polyclonal immunoglobulin composition by collecting the fraction of a human polyclonal immunoglobulin composition on an affinity chromatography column carrying ligands that specifically recognize anti-A and anti-B antibodies.

### Example 2

### Preparation of Human Polyclonal Immunoglobulin Compositions Enriched in Human Anti-A Polyclonal Immunoglobulins or in Human Anti-B Polyclonal Immunoglobulins

The Inventors next sought to separate human anti-A polyclonal immunoglobulins from human anti-B polyclonal immunoglobulins.

Materials and methods

In order to separate human anti-A polyclonal immunoglobulins from human anti-B polyclonal immunoglobulins, two aliquots of the composition obtained in Example 1 were subjected to two different treatments:
1. In order to obtain a purified human anti-A polyclonal immunoglobulin composition, a first aliquot was adsorbed on a 1 ml affinity chromatography column filled with gel consisting of porous cross-linked cellulose beads grafted with the trisaccharide characteristic of group B antigens (column 1). The load was 1.2 mg of purified human anti-A and anti-B polyclonal immunoglobulin composition per ml of gel. Contact time was set to 2 minutes.
The unadsorbed fraction was collected for subsequent analyses.
2. In order to obtain a purified human anti-B polyclonal immunoglobulin composition, a second aliquot was adsorbed on a 1 ml affinity chromatography column filled with gel consisting of porous cross-linked cellulose beads grafted with the trisaccharide characteristic of group A antigens (column 2). The load was 1.2 mg of purified human anti-A and anti-B polyclonal immunoglobulin composition per ml of gel. Contact time was set to 2 minutes.
The unadsorbed fraction was collected for subsequent analyses.

Results

Results obtained are summarized in Table 3 below:

TABLE 3
IgG concentration and anti-A and anti-B
activities of the unadsorbed fraction.

| | Column 1 | Column 2 |
|---|---|---|
| | Unadsorbed fraction | Unadsorbed fraction |
| IgG (mg/L) | 3.4 | 3.6 |
| Anti-A (AU*) | 7323 | 0.00 |
| Anti-B (AU*) | 0.00 | 4248 |

*AU = arbitrary units with respect to the lyophilized human normal immunoglobulin medicament set to 1.

Results presented in Table 3 show that all of the anti-B immunoglobulins are adsorbed on column 1, as no anti-B activity is detected in the unadsorbed fraction. The latter has only very high anti-A activity. Indeed, compared with the anti-A activity of 622.3 AU of the composition obtained in Example 1, the unadsorbed fraction on column 1 has anti-A activity of 7323 AU, which is about 11.8 times superior to that of the composition obtained in Example 1.

Thus, passing the composition obtained in Example 1 on the column (grafted with the trisaccharide that is characteristic of B antigens) makes it possible to obtain a purified anti-A immunoglobulin composition by collecting the non-adsorbed fraction.

Similarly, the results presented in Table 3 show that all of the anti-A immunoglobulins are adsorbed on column 2, as no anti-A activity is detected in the unadsorbed fraction. The latter has only very high anti-B activity. Indeed, compared with the anti-B activity of 638.7 AU of the composition obtained in Example 1, the unadsorbed fraction on column 2 has an anti-B activity of 4248 AU, which is about 6.7 times superior to that of the composition obtained in Example 1.

Thus, passing the composition obtained in Example 1 on the column 2 (grafted with the trisaccharide that is characteristic of A antigens) makes it possible to obtain a purified anti-B immunoglobulin composition by collecting the unadsorbed fraction.

Conclusions

The results presented above show that the use of a second affinity chromatography step with a column carrying the trisaccharide characteristic of A antigens or the trisaccharide characteristic of B antigens makes it possible to separate anti-A immunoglobulins from anti-B immunoglobulins, and thus to obtain purified anti-A immunoglobulin or anti-B immunoglobulin compositions.

The purified anti-A immunoglobulin composition is obtained by collecting the fraction that is not adsorbed on a column carrying the trisaccharide characteristic of B antigens. Indeed, the operating conditions are optimised in such a way that all of the anti-B immunoglobulins bind to a column bearing the trisaccharide characteristic of B antigens. In the same way, the purified anti-B immunoglobulin composition is obtained by collecting the fraction that is not adsorbed on a column carrying the trisaccharide characteristic of A antigens. Indeed, the operating conditions are optimised in such a way that all of the anti-A immunoglobulins bind to a column bearing the trisaccharide characteristic of A antigens.

To obtain a purified anti-A immunoglobulin composition from the composition obtained in Example 1, it is thus necessary to collect the unadsorbed fraction from a column carrying the trisaccharide characteristic of B antigens.

In the same way, to obtain a purified anti-B immunoglobulin composition from the composition obtained in Example 1, it is thus necessary to collect the unadsorbed fraction from a column carrying the trisaccharide characteristic of A antigens.

The purified anti-A immunoglobulin and/or anti-B immunoglobulin compositions may be used as a positive control in a method for assaying the anti-A and/or anti-B activity of a human polyclonal immunoglobulin composition.

### Example 3

### Characterization of Human Polyclonal Immunoglobulin Compositions, Enriched in Human Anti-A Polyclonal Immunoglobulins or in Human Anti-B Polyclonal Immunoglobulins, Obtained in Example 2

The human anti-A and anti-B polyclonal immunoglobulin composition obtained by elution of the adsorbed fraction of a first chromatography step in Example 1 was concentrated by ultrafiltration on a Pellicon 2 membrane, 30 kDa, 0.1 m² (Merck Millipore®). The human anti-A polyclonal immunoglobulin or human anti-B polyclonal immunoglobulin compositions (unadsorbed fractions obtained at the conclusion of the second chromatography step of Example 2) were concentrated by ultrafiltration using a Pellicon XL Biomax membrane, 30 kDa, 50 cm² (Merck Millipore®) then reconcentrated 10-fold by centrifugation on Amicon Ultra, Ultracel membrane (Merck Millipore®). The compositions are then characterized in greater detail with regard to the distribution of various IgG subclasses, integrity of the IgGs (percentages of monomers, dimers, polymers, and fragments), and their activity with respect to blood group O red blood cells (carrying neither A antigens nor B antigens), blood group A red blood cells (carrying A antigens, but not B antigens), and blood group B red blood cells (carrying B antigens, but not A antigens). The same characterization was carried out on a human normal immunoglobulin composition administered intravenously (IgIV) depleted of human anti-A and anti-B polyclonal immunoglobulins.

Characterization Methods

IgG Subclass Distribution

The distribution of IgG subclasses was measured by three different methods: nephelometry, mass spectrography, and electroluminescence (MSD).

Nephelometry: The principle of the nephelometry assay is based on the liquid-phase reaction of an antigen with a specific antibody. The insoluble antibody-antigen complex formed causes turbidity, which is measured by a nephelometric technique, whose principle is as follows: when a light beam passes through a turbid medium, a portion of the light is deflected from its trajectory (scattering phenomenon). Measurement by means of a photoreceptor of light deflected at a certain angle with regard to the incident beam makes it possible to quantify turbidity via an electrical signal. The relationship between antigen concentration and the electrical signal obtained, for a constant antibody concentration, is described by the Heidelberger-Kendall curve. The measuring range located in the ascending portion of the curve, corresponding to excess antibody. The nephelometry measurements were made with a BNII nephelometer (Siemens) with software version 2.5/F.

Mass spectrography (LC-MS): This method permits the quantification and characterization of IgG subclasses and allotypes. Cysteine proteinase of Streptococcus pyogenes (IdeS) specifically cleaves human IgG in the hinge region. Fc/2 fragments generated from the IgG subclasses are separated by chromatography and the various allotypes of each subclass are characterized by mass spectrometry.

Enzymatic proteolysis and glycolysis were obtained by incubating 10 μl of sample at a concentration of 10 mg/ml in phosphate buffered saline with 100 IU of IdeS and 100 IU of IgGZERO enzymes for 1 hour at 37° C. Reduction of digested IgGs was carried out by adding 35 μl of denaturing buffer (8 M guanidine-HCl, 0.1 M Tris-HCl, pH 7.5) and 5 μl of 200 mM DTT solution. The preparation was incubated at 50° C. for 30 minutes.

Separation of Fc/2 fragments was carried out on an ACQUITY system (Waters, Milford, Mass., USA) coupled to a UV detector and an electrospray mass spectrometer (Synapt G2S, Waters, Milford, Mass., USA). About 20 μg of sample was injected on a Pursuit diphenyl 150 mm×2.0 mm column (Agilent, Santa Clara, Calif., USA) equilibrated at 70° C. and operating with a 200 μl/min flow rate. An elution gradient was applied using a solvent A (0.1% TFA in water) and a solvent B (90% MeCN, 10% water and 0.1% TFA), after isocratic elution with 10% of B for 5 minutes, B was brought to 38% for 10 minutes and to 44% for an additional 48 minutes. The column was then washed for 4 minutes with 80% of B, and in addition equilibrated for 9 minutes with 10% of B. The mass spectrometer was used in positive resolution mode and the data were recorded for m/z from 200 to 2000. The protein mass spectra were deconvoluted using the MassLynx software.

Electroluminescence (MSD): Detection by electroluminescence uses labels that emit light when they are stimulated electrochemically. Background noise is minimal because the stimulation mechanism (electricity) is decoupled from the signal (light). The labels are stable, non-radioactive and emit light at −620 nm, eliminating quenching problems. Few compounds interfere with electroluminescent labels. Multiple excitation cycles of each label amplify the light signal level and increase sensitivity. Meso Scale Discovery (MSD) technology was used, according to the manufacturer's recommendations. This technology is based on the use of microplates comprising carbon electrodes integrated into the bottom of the wells.

Integrity of the IgGs

Quantification of the polymer, dimer and monomer forms, and of possible breakdown products (fragments), of the IgGs was measured by two diffrent methods: SDS-PAGE and high-performance size-exclusion chromatography (HPSEC).

SDS-PAGE: The samples were analyzed by electrophoretic migration on an SDS-PAGE gel in a Multiphor system (GE). 2 μg of sample is added to 3 μl of 250 mM Tris, pH 7.5, 5% SDS buffer (+1.5 μl of NuPAGE Sample Reducing Agent (10X)—Invitrogen ref. NP0004 for the reducing conditions). After shaking, the sample is heated at 95° C. for 3 minutes just before loading on an ExcelGel SDS Homogeneous 12.5% gel. The following migration protocol is used:
for 1 whole gel: 600 V; 50 mA; 30 W for 1.5 hours;
for 2 whole gels: 600 V; 100 mA; 60 W for 1.5 hours.

For developing with CBB (Coomassie Brilliant Blue, BioRad ref. 161-0406), the gel is placed in contact with staining solution (50% methanol, 7% acetic acid, 0.1% CBB) with shaking for 30 minutes, then with destaining solution 1 (50% methanol, 7% acetic acid) with shaking for 5 minutes, then destaining solution 2 (5% methanol, 7% acetic acid) with shaking until a clear and homogeneous background is obtained. When the gel is sufficiently destained, it is placed in water.

For developing with silver nitrate, the gel is placed in contact with fixing solution (250 ml of 40% ethanol, 10% acid acetic in WFI), with moderate shaking on a shaker for 30 minutes, then with sensitizing solution (75 ml of absolute ethanol+17 g of sodium acetate+10 ml of sodium thiosulfate, q.s. to 250 ml with WFI+1.25 ml of glutaraldehyde immediately before use) with shaking for 30 minutes, before being rinsed 3×5 minutes in WFI. The gel is then brought into contact with AgNO₃ solution (10% AgNO₃ in 250 ml of WFI+100 μl of formaldehyde immediately before use) with shaking for 20 minutes, before being rinsed 2×1 minute in WFI. The gel is then brought into contact with developer solution (6.25 g of sodium carbonate in 250 ml of WFI+50 μl of formaldehyde immediately before use), and staining is left to develop (2 to 5 minutes maximum) with shaking. Development is stopped by discarding the solution and placing the gel in contact with EDTA solution (3.65 g of EDTA in 250 ml of WFI) for 10 minutes minimum.

HPSEC: This technique makes it possible to separate proteins by molecular size. A Dionex UltiMate 3000 system with a Superdex Tricorn 200 10/300 GL column (GE Healthcare ref. 17-5175-01) was used. The following analysis conditions were used:
Flow rate: 0.4 ml/min
Mobile phase: PBS buffer
Injector temperature: 5° C. 0 2° C.
Column temperature: room temperature
Detection wavelength: 280 nm
Volume injected: cf. § 5.3.1, 5.3.2, 5.3.3
Acquisition time: 60 minutes

The chromatograms are integrated and relative areas are calculated by the Chromeleon software in %, according to the formula:

Relative peak area Y=(peak area Y×100)/total area

Activity of the Compositions

The assay of anti-A or anti-B activity on A, B or O red blood cells was carried out by cytometry test using a F(ab′)₂ labeled with a fluorescent label, as described in example 2 of application WO2007/077365.

Briefly, a suspension of red blood cells at a concentration of 40·10⁶ red blood cells/ml is prepared in 1% PBS/BSA. 50 μl of this suspension is deposited in a 96-well round-bottom microplate (2·10⁶ red blood cells/well). Dilution ranges of the standards (lyophilized human normal immunoglobulin or EDQM (European Directorate for the Quality of Medicines Et HealthCare) reference standard Y0001688) are prepared. 50 μl of a dilution of a standard or of a sample to be tested is added to the 50 μl of red blood cells in the microplate, which is covered with an adhesive film and incubated for 2 hours at 37° C. ±2° C. with shaking. After three washes in 1% PBS/BSA, conjugated goat F(ab′)₂ anti-human labeled with a fluorescent label is added and the plate is incubated for 20 to 30 minutes at room temperature shielded from light. After 3 washes, dilutions of the standard and of the samples are taken up in 200 μl of 1% PBS/BSA. The samples are then analyzed by flow cytometry (Beckman Coulter FC500). Reading is carried out on 50,000 events and the device automatically calculates mean fluorescence intensity (MFI) of each point of the range or sample. MFI is plotted as a function of the concentration of the standard and the equation of the regression line is obtained by means of the Excel software. Then, for each sample, the concentration in standard equivalents is obtained using the linear equation of the regression line.

Activity is expressed in arbitrary units with respect to the standard (EDQM reference standard Y0001688 or medicament (lyophilized human normal immunoglobulin)) set to 1.

Results

Distribution of IgG Subclasses

Results are presented in Table 4 below, and show that the human anti-A and anti-B polyclonal immunoglobulins obtained in Example 1, after elution of the fraction of total human polyclonal immunoglobulin adsorbed on an affinity chromatography column carrying the trisaccharide characteristic of group A antigens and the trisaccharide characteristic of group B antigens, are enriched in IgG2 when compared with the human polyclonal immunoglobulin composition depleted of human anti-A and anti-B polyclonal immunoglobulins. This is also the case, in an even more pronounced manner, for the human anti-A polyclonal immunoglobulin or human anti-B polyclonal immunoglobulin compositions (unadsorbed fractions obtained at the conclusion of the second chromatography step of Example 2). Thus, the human anti-A polyclonal immunoglobulins and the human anti-B polyclonal immunoglobulins of isotype IgG are characterized by enrichment in isotype IgG2.

TABLE 4
Concentration and distribution of IgG subclasses among the various compositions tested

| | Human normal immunoglobulin | | | | | Human anti-A and anti-B | | | | |
|---|---|---|---|---|---|---|---|---|---|---|
| Reference | composition depleted of human anti-A and | | | | | polyclonal immunoglobulin | | | | |
| products | anti-B polyclonal immunoglobulins | | | | | composition (Example 1) | | | | |
| IgG (g/l) | 50 | | | | | 14.4 | | | | |
| | Nephelometry | | Spectrography | MSD | | Nephelometry | | Spectrography | MSD | |
| IgG1 (g/l) | 29.6 | (61%) | 65% | 35.44 | (65%) | 4.42 | (32.6%) | 28% | 4.74 | (40.6%) |
| IgG2 (g/l) | 17.1 | (35%) | 32% | 14.82 | (27%) | 8.82 | (65.1%) | 70% | 6.06 | (52%) |
| IgG3 (g/l) | 1.049 | (2.16%) | 3% | 3.54 | (6.48%) | 0.227 | (1.7%) | 2% | 0.851 | (7.3%) |
| IgG4 (g/l) | 0.744 | (1.5%) | | 0.823 | (1.5%) | 0.0807 | (0.59%) | | 0.068 | (0.58%) |
| Sum (g/l) | 48.493 | | | 54.623 | | 13.5477 | | | 11.6578 | |
| | Human anti-A polyclonal | | | | | Human anti-B polyclonal | | | | |
| Reference | immunoglobulin composition | | | | | immunoglobulin composition | | | | |
| products | (Example 2) | | | | | (Example 2) | | | | |
| IgG (g/l) | 0.694 | | | | | 0.538 | | | | |
| | Nephelometry | | Spectrography | MSD | | Nephelometry | | Spectrography | MSD | |
| IgG1 (g/l) | 0.0904 | (17.6%) | 15% | 0.049 | (18.8%) | 0.127 | (19%) | 16% | 0.046 | (19%) |
| IgG2 (g/l) | 0.408 | (79.6%) | 84% | 0.197 | (75.6%) | 0.474 | (72.2%) | 81% | 0.175 | (73.6%) |
| IgG3 (g/l) | 0.00264 | (0.51%) | 2% | 0.0068 | (2.6%) | 0.00432 | (0.65%) | 3% | 0.00909 | (3.8%) |
| IgG4 (g/l) | 0.01156 | (2.25%) | | 0.00783 | (3%) | 0.01185 | (1.8%) | | 0.00752 | (3.16%) |

| | | | | |
|---|---|---|---|---|
| Sum (g/l) | 0.5126 | 0.2606 | 0.65605 | 0.23761 |

Integrity of the IgGs

SDS-PAGE and HPSEC results are summarized in Table 5 below. Results obtained show that more than 87% of the immunoglobulins are intact for all the compositions tested.

TABLE 5
Integrity of immunoglobulins in the compositions tested.

| | | Human anti-A and anti-B |
|---|---|---|
| Reference | | polyclonal immunoglobulin |
| products | | composition (Example 1) |
| | | SDS-page |
| IgG integrity | | 89.60% |
| | | HPSEC |
| Polymers 2 | | — |
| Polymers 1 | | 0.50% |
| Dimers | | 7.30% |
| Monomers | | 90.90% |
| Fragments | | 1.30% |
| | Human anti-A polyclonal | Human anti-B polyclonal |
| Reference | immunoglobulin composition | immunoglobulin composition |
| products | (Example 2) | (Example 2) |
| | SDS-page | SDS-page |
| IgG integrity | 87.00% | 87.80% |
| | HPSEC | HPSEC |
| Polymers 2 | 0.70% | — |
| Polymers 1 | 0.50% | 8.30% |
| Dimers | 3.00% | 3.10% |
| Monomers | 94.40% | 87.30% |
| Fragments | 1.30% | 1.40% |

ND: not determined.

Activity with Respect to Red Blood Cells

Results are presented in Table 6 below, expressed in arbitrary units with respect to the EDQM reference standard or to lyophilized human normal immunoglobulin medicament, set to 1.

TABLE 6
Activity of compositions tested in the cytometry test
with respect to blood group O, A and B red blood cells.

| | Human normal immunoglobulin | | Human anti-A and anti-B | |
|---|---|---|---|---|
| Reference | composition depleted of human anti-A and | | polyclonal immunoglobulin | |
| product | anti-B polyclonal immunoglobulins | | composition (Example 1) | |
| | | Lyophilized | | Lyophilized |
| | | human normal | | human normal |
| | | immunoglobulin | | immunoglobulin |
| Cytometry | EDQM ref. | ref. | EDQM ref. | ref. |
| A red blood | 0.17 | 0.75 | 251.36 | 930.24 |
| cell activity | | | | |
| B red blood | 0.07 | 0.43 | 344.06 | 1271.81 |
| cell activity | | | | |
| O red blood | 0 | 0 | 0 | 0 |
| cell activity | | | | |
| Anti-A:anti-B | 2.43 | 1.74 | 0.73 | 0.73 |
| ratio | | | | |
| Anti-B:anti-A | 0.41 | 0.57 | 1.37 | 1.37 |
| ratio | | | | |
| | Human anti-A polyclonal | | Human anti-B polyclonal | |
| Reference | immunoglobulin composition | | immunoglobulin composition | |
| product | (Example 2) | | (Example 2) | |
| | | Lyophilized | | Lyophilized |
| | | human normal | | human normal |
| | | immunoglobulin | | immunoglobulin |
| Cytometry | EDQM ref. | ref. | EDQM ref. | ref. |
| A red blood | 2801.73 | 11043.7 | 0 | 0 |
| cell activity | | | | |
| B red blood | 0 | 0 | 4923.24 | 19326.71 |
| cell activity | | | | |
| O red blood | 0 | 0 | 0 | 0 |
| cell activity | | | | |

*AU = arbitrary units with respect to the EDQM reference standard or to lyophilized human normal immunoglobulin medicament set to 1.

These results confirm that it is possible to obtain, by the methods described herein, purified compositions:
highly enriched in human anti-A polyclonal immunoglobulins and having high anti-A activity but no anti-B activity, or
highly enriched in human anti-B polyclonal immunoglobulins and having high anti-B activity but no anti-A activity.

### BIBLIOGRAPHIC REFERENCES

Thorpe S J, Fox B J, Dolman C D, Thorpe R. Anti-A and anti-B activity in batches of different intravenous immunoglobulin products determined using a direct haemagglutination method. Biologicals. 2005 June; 33(2):111-6.

Thorpe S J, Fox B, Sharp G, Heath A B, Behr-Gross M E, Terao E, Virata-Theimer M L, Yu M W. International collaborative study to evaluate candidate reference reagents to standardize haemagglutination testing for anti-A and anti-B in normal intravenous immunoglobulin products. Vox Sang. 2009 August; 97(2):160-8.

Thorpe S J, Fox B, Sharp G, Heath A B, Behr-Gross M E, Terao E, Virata-Theimer M L, Yu M W. International collaborative study to establish reference preparations to standardise haemagglutination testing for anti-A and anti-B in normal intravenous immunoglobulins by the direct method. Pharmeur Bio Sci Notes. 2010 April;2010(1):39-50.

WHO/BS/08.2091

WO99/64462

WO01/27623

WO02/092632

WO2007/077365

1. A composition comprising human polyclonal immunoglobulins, wherein at least 80% by weight of human polyclonal immunoglobulins present in the composition are human anti-A and/or anti-B polyclonal immunoglobulins.

2. The composition according to claim 1, wherein human polyclonal immunoglobulins represent at least 85% by weight of the total proteins of the composition.

3.-4. (canceled)

5. The composition according to claim 1, wherein it has an anti-A or anti-B activity enriched by a factor of at least 4 as compared to a lyophilized normal human immunoglobulin reference or an EDQM reference.

6. The composition according to claim 1, wherein the composition comprises both human anti-A polyclonal immunoglobulins and human anti-B polyclonal immunoglobulins, and wherein the weight ratio of human anti-A polyclonal immunoglobulins to human anti-B polyclonal immunoglobulins is between 1:10 and 10:1.

7. The composition according to claim 1, wherein it has a ratio (anti-A activity:anti-B activity) between 1:10 and 10:1.

8. The composition according to claim 6, wherein the weight ratio of human anti-A polyclonal immunoglobulins to human anti-B polyclonal immunoglobulins is between 2:1 and 10:1.

9.-10. (canceled)

11. The composition according to claim 1, wherein it has a human anti-A and/or anti-B polyclonal immunoglobulin concentration superior to 1 g/l.

12. The composition according to claim 1, wherein at least 90% by weight of human polyclonal immunoglobulins present in the composition are IgGs.

13. The composition according to claim 12, wherein at least 40% by weight of the IgG-isotype human polyclonal immunoglobulins present in the composition are of subclass IgG2.

14. The composition according to claim 1, wherein it has an IgG2:IgG1 weight ratio of at least 0.8.

15. A method for preparing a composition according to claim 1, comprising the following steps:
a) adsorbing a batch of human plasma or a human plasma fraction enriched in human polyclonal immunoglobulins on a support grafted with a specific ligand of human anti-A polyclonal immunoglobulins and/or with a specific ligand of human anti-B polyclonal immunoglobulins, to form an adsorbed fraction and an unadsorbed fraction,
b) storing the unadsorbed fraction for possible later use, and
c) dissociating and collecting the adsorbed fraction.

16. The method for preparing a composition according to claim 15, further comprising the following steps:
d) adsorbing the composition obtained in step c) on a support grafted with a specific ligand of human anti-B polyclonal immunoglobulins or adsorbing the composition obtained in step c) on a support grafted with a specific ligand of human anti-A polyclonal immunoglobulins, and
e) collecting the unadsorbed fraction,
wherein when a specific ligand of human anti-B polyclonal immunoglobulins is used in step d),
wherein the composition comprises at least 80% by weight of human anti-A polyclonal immunoglobulins, and
wherein when a specific ligand of human anti-A polyclonal immunoglobulins is used in step d), the composition comprises at least 80% by weight of human anti-B polyclonal immunoglobulins.

17. (canceled)

18. The method according to claim 15, wherein the specific ligand of human anti-A polyclonal immunoglobulins comprises trisaccharide GalNAcα1-3(Fucα1-2)Gal.

19. The method according to claim 15, wherein the specific ligand of human anti-B polyclonal immunoglobulins comprises trisaccharide Galα1-3(Fucα1-2)Gal.

20. The method according to claim 15, wherein the support used in step a) and/or in step d) is in the form of:
a) particles grafted with the ligand(s) of interest, or
b) a polymer membrane, the membrane being grafted with the ligand(s) of interest.

21. The method according to claim 20, wherein the particle polymer or the membrane polymer is selected from the group consisting of cellulose and derivatives thereof, agarose, dextran, polyacrylates, polystyrene, polyacrylamide, polymethacrylamide, styrene and divinylbenzene copolymers, and mixtures of these polymers.

22. The method according to claim 21, wherein the ligand(s) of interest is(are) grafted on the polymer particles or the polymer membrane via a spacer.

23. (canceled)

24. The method for preparing a composition according to claim 15,
wherein the human plasma fraction of step a) is a pre-purified fraction of human polyclonal immunoglobulins, said fraction being adsorbed on a support grafted with a specific ligand of human anti-B polyclonal immunoglobulins, and with a specific ligand of human anti-A polyclonal immunoglobulins, the unadsorbed fraction of step b) is collected for use in a method of purifying a concentrate of human polyvalent immunoglobulins depleted of human anti-A and anti-B polyclonal immunoglobulins, and
wherein the method further comprises the following steps:
d) adsorbing a part of the composition obtained in step c) on a support grafted with a specific ligand of immunoglobulins recognizing blood group B antigens, and collecting the unadsorbed fraction enriched in anti-A, and
e) adsorbing the other part of the composition obtained in step c) on a support grafted with a specific ligand of immunoglobulins recognizing blood group A antigens, and collecting the unadsorbed fraction enriched in anti-B.

25. A method for assaying anti-A and/or anti-B activity of a composition comprising human polyclonal immunoglobulins, wherein said method comprises using the composition according to claim 1 as a positive control.

26.-29. (canceled)
